(19) United States
(12) Patent Application Publication
(10) Pub. No.: US 2011/0311568 A1
(43) Pub. Date: Dec. 22, 2011

(54) Vaccine

(76) Inventors: Veronica Fowler, Berkshire (GB); Paul Barnett, Berkshire (GB); Nick Knowles, Berkshire (GB)

(21) Appl. No.: 12/997,846

(22) PCT Filed: Jun. 11, 2009
(86) PCT No.: PCT/GB2009/001469

§ 371 Date: Mar. 11, 2011

(30) Foreign Application Priority Data
Jun. 11, 2008 (GB) 0810710.4

(51) Int. Cl. A61K 39/135; A61P 31/14; A61P 37/04; C12Q 1/70; C40B 30/04

(52) U.S. Cl. 424/186.1; 435/5; 506/9

(57) ABSTRACT

The present invention provides a foot and mouth disease (FMD) vaccine comprising or capable of expressing afoot and mouth disease virus (FMDV) VP1 polypeptide having a deletion of at least seven amino acids in the G-H loop such that the VP1 polypeptide lacks an RGD motif. The present invention also provides a method for preventing and/or treating FMD in a subject which comprises the step of administration of such a vaccine.

### FIELD OF THE INVENTION

The present invention relates to a vaccine for preventing and/or treating foot and mouth disease (FMD). The vaccine comprises a vaccinating entity which comprises, or is capable of, expressing a foot and mouth disease virus (FMDV) VP1 polypeptide having a deletion in the G-H loop.

### BACKGROUND TO THE INVENTION

### Foot and Mouth Disease (FMD)

FMD is a highly contagious and economically devastating disease of cloven-hoofed animals (Artiodactyla), affecting domesticated ruminants, pigs and a large number of wildlife species (Alexandersen et al., (2003) Journal of Comparative Pathology. 129. 1-36.) of which the causal agent is Foot-and-Mouth Disease Virus (FMDV).

FMDV is a positive sense, single stranded RNA virus and is the type species of the Aphthovirus genus of the Picornaviridae family. FMDV exists as seven antigenically distinct serotypes namely A, O, C, Asia 1 and South African Territories (SAT) 1, 2 and 3, with numerous subtypes within each serotype. With the exception of New Zealand, outbreaks have been recorded in every livestock-containing region of the world and the disease is currently enzootic in all continents except Australia and North America. Although mortality rates are generally low (less than 5%) in adult animals, the UK 2001 FMD Pan-Asian O outbreak clearly identifies the serious economic consequences associated with the disease, with the cost to the public sector estimated at over 4.5 billion euros and the cost to the private sector at over 7.5 billion euros (Royal Society Report, (2002), on Infectious Disease in Livestock-Scientific questions relating to the transmission, prevention and control of epidemic outbreaks of infectious disease in livestock in Great Britain. (2002) Latimer Trend Limited, Cornwall, UK.).

FMD is ranked first in the l'Office International des Epizooties (OIE, World Organisation for Animal Health) list of notifiable diseases, which by definition, means that it has the potential for rapid and extensive spread within and between countries. Thus, current intensive farming practices and high stocking densities clearly encourage the rapid spread of such a disease.

FMD is widely distributed throughout the world. Developed regions such as the continents of North and Central America and Antarctica, and countries such as Australia and New Zealand are free from disease while FMD is endemic in many developing countries such as those in sub-Saharan Africa, the Middle East, southern Asia, Southeast Asia and South America (FIG. 1.2). There are also some areas of the world which are normally free from disease, such as Europe where FMD was eradicated in 1989 and where vaccination has ceased since 1991. However, there have been occasional incursions of disease such as the 2001 UK/EIRE/France/Netherlands epidemic due to a PanAsian O strain (Knowles et al., (2001) Veterinary Record. 148. 258-259) and the 2007 UK outbreak of serotype O1 BFS/1967.

### Carrier Status

It is thought that, with the possible exception of pigs and some wild hosts, all animals which are susceptible to infection with FMDV have the capacity to become asymptomatic carriers of FMDV. Persistence that leads to the carrier status is defined as the ability to recover virus from oesophageal-pharyngeal fluid 28 days or more post infection (Kitching, (2002) Review of Science and Technology. 21. 531-8.). Persistence can occur in both vaccinated and non-vaccinated animals (Doel et al., (1994) Vaccine. 12. 592).

The prevalence rate of carriers depends on a number of variables including the species, incidence of disease and the immune status of the population. Carrier status in African Buffalo has been reported to be as high as 50-70% (Hedger, (1972) Journal of Comparative Pathology. 82. 1, 19-28), although for cattle and sheep the prevalence rate has rarely exceeded 50% (Anderson et al., (1976) Journal of Hygiene (London). 76. 395-402). The duration of the carrier state varies between species, African buffalo have been reported to excrete virus for at least 5 years (Condy et al., (1985) Comparative Immunology and Microbiology of Infectious Diseases. 8. 259-265), sheep for up to one year, while goats are reported to excrete virus for up to four months (Salt, (2004) Horizon Bioscience. 103-143).

Carrier status poses many problems for vaccination, as the carrier state can appear in vaccinated individuals, depending on the antigen payload and type of exposure (Doel et al., (1994) as above, Barnett et al., (2004) Vaccine. 22. 1221-1232. and Cox et al., (2006) Vaccine. 24. 3184-3190). The conventional vaccine, although providing sufficient protection to prevent clinical disease, is not known to induce sterile immunity and therefore virus replication can still occur in some animals not showing clinical signs. There is thus the perceived risk that the animals, which fail to clear the virus and subsequently become persistently infected, may transmit disease to other susceptible livestock.

Although there is no conclusive evidence that carriers can transmit disease, even under experimental conditions (Kitching, (2002) as above, Panda et al., (2006) Vaccine. 24. 964-969 and Moonen et al., (2004) Veterinary Microbiology. 103. 151-160), the potential risk is sufficient to have had a major impact on international trade in livestock and their products, and on the decision whether or not to use vaccines to assist in the control of an FMD outbreak.

Currently, the World Organisation for Animal health recognizes countries to be in one of three disease states with regards to FMD: FMD present with or without vaccination, FMD-free with vaccination and FMD-free without vaccination. Countries such as the UK that are designated FMD-free without vaccination have the greatest access to export markets, and therefore are anxious to maintain their current status.

The non-vaccination approach used in some FMD-free countries, such as the UK, involves a stamping out policy, combining slaughter of infected and in contact animals with restrictions on animal movements, surveillance and epidemiological tracing should an outbreak occur. This control strategy is, however, very much dependent on early diagnosis and co-operation of livestock owners.

Moreover, the use of emergency vaccination to control an outbreak is complicated by the need to be able to detect sub-clinically infected carriers with a high certainty amidst large numbers of vaccinates and the consequential need for extremely sensitive and specific serological tests to detect infection-specific antibodies. The public outcry following the mass slaughter of animals as a result of the 2001 UK FMD epidemic stimulated a review of control policy and encouraged the use of vaccination as a principal means of control. A new EU Directive on the control of FMD was introduced in 2003, placing increased emphasis on the consideration of a vaccinate-to-live policy and therefore, it has now become a priority to develop improved vaccines, alongside refined diagnostics which permit detection of sub-clinical infection in vaccinated animals, in order for emergency vaccination to be a feasible option in FMD-free countries.

In the absence of a vaccine offering long term sterile immunity (which would abolish the concern over the carrier status), it is highly desirable to develop a “marker vaccine” enabling discrimination between vaccinated, infected and vaccinated/subclinically infected animals.

### Currently Available FMD Vaccines

Conventional vaccines against FMD consist of whole virus virions that have been chemically inactivated, normally by use of an aziridine such as binary ethyleneimine (BEI). More than a billion doses are used annually worldwide (Rweyemamu and Le Forban (1999) Advances in Virus Research. 53. 111-126) and in many countries have been utilised successfully in controlling the disease.

In the absence of specifically engineered marker vaccines, the approach to FMD differentiation has centred on serological assays, such as ELISAs for detection of non-structural proteins (NSPs). Conventional vaccines, following appropriate purification steps, should lack NSP's such as 3ABC, 3AB and 2C (Clavijo et al., (2004) The Veterinary Journal. 167. 9-22; Bergmann et al., (2000) Archives of Virology. 145. 473-489; Brocchi et al., (1998) Veterinary Quarterly. 2. 20-24; Mackay et al., (1998) Veterinary Quarterly. 20. Supplement 2, S9-11).

There are three commercially available tests for antibodies to FMD NSP, the FMD-CHEKIT-ELISA (Bommeli) and the Ceditest® (Cedi Diagnostics B.V.) which utilises recombinant 3ABC antigen, and the UBI FMDV NSP ELISA which utilises a peptide representing 3B. Bergmann et al. (1993) (American Journal of Veterinary Research. 54. 825-31) also developed the use of the enzyme-linked immunoelectrotransfer blot test (EITB) which utilises five non-capsid recombinant antigens as serological probes as an alternative means to identify NSP. The theory is that in the absence of NSP's in highly purified conventional vaccines, vaccinated animals which subsequently become infected will, as a result of supporting live virus replication, produce an NSP antibody response. However, this practice has met with varying degrees of success, including variation between species and NSP test. NSP assays are more consistent in cattle compared to sheep where the frequent sub-clinical nature of disease may fail to induce detectible levels of NSP (Kitching, (2004 Diagnosis of Foot-and-Mouth Disease. In Foot and Mouth Disease Current Perspectives. Edited by Francisco Sobrino and Esteban Domingo. Horizon Biosciences. 411-424). Panda et al. ((2005) Vaccine. 23. 5186-5195) and Bronsvoort et al. ((2004) Journal of Clinical Microbiology. 42. 2108-2114), found inconsistencies in the ability of commercially available serological tests to detect antibodies to NSP at the individual animal level. For example, Panda et al. ((2005) as above) reported that the Ceditest FMDV-NS ELISA could identify 7/9 animals as persistently infected, whereas the Bommeli CHEKIT-FMD-3ABC ELISA and UBI FMDV 3B peptide NSP ELISA could only identify 5/9 animals as persistently infected. In addition the studies by Bronsvoort et al. ((2004) as above) reported the Bommeli CHEKIT-FMD-3ABC ELISA to be only 23% sensitive, due to its ability to only detect 47% of known carrier animals. These results indicate that such tests may have difficulties in detecting a low prevalence of carrier animals, due to the great variability in the initiation, specificity and duration of the immune response in individual animals to the NSPs, thus limiting their use to herd level diagnosis (reviewed in Clavijo et al., (2004) as above).

In addition to this, vaccine preparations, depending on their source, can on occasion contain traces of NSP, reducing the specificity of the assay (Mackay et al., (1998) as above). Some vaccinated animals exposed to infection can also become asymptomatic carriers, without the associated 3ABC NSP seroconversion (Mackay et al., (1998) as above; Sorensen et al., (1998) Archives of Virology. 143. 1461-1476).

There is thus a need for an improved FMD vaccine, which coupled with an appropriate diagnostic assay, allows a more definitive distinction between vaccinated and infected individuals.

### Immunodominance of the VP1 G-H Loop

Early work showed that, out of all the individual coat proteins, only VP1 was capable of inducing a neutralising antibody response (Laporte et al., (1973) Comples Rendues Hebdomadaires des Seances de l'Academie des sciences. Serie D. 276. 2299-3402 and Bachrach et al., (1975) Journal of Immunology. 115. 6, 1636-41, although referred to as VP3 at that time). Proteolytic cleavage studies refined this work further, identifying the immunodominant site of FMDV to be that of the G-H loop of VP1 (Site 1) (Strohmaier et al., (1982) Journal of General Virology. 59. 295-306, Bachrach et al., (1975) as above, Wild et al., (1969) Journal of General Virology. 3. 313-320, and Wild and Brown, (1967) Journal of General Virology. 1. 247-250).

It is now widely accepted that the VP1 G-H loop is a known immunodominant site of the FMD virus. This view is supported by observations such as the following, i) a large number of MAbs recognise the VP1 G-H loop either continuously or discontinuously, ii) trypsin treatment, which is known to cleave the VP1 G-H loop, results in reduced antigenicity and immunogenicity of the virus, iii) up to 60% of virus neutralising antibody is directed to the VP1 G-H loop in infected animals (Mateu et al., (1995) Virology. 206. 298-306), and iv) VP1 G-H loop peptides induce strong levels of virus neutralising antibody which in some species is protective.

In a study investigating alternatives to chemical inactivation, integrity of the capsid was evaluated using antibodies against epitopes on the surface of the virus. Great importance was attached to the fact that the integrity of the VP1 G-H loop was not compromised (Suryanarayana et al (2002) Vaccine 20:1163-1168).

There is thus a widespread belief that the VP1 G-H loop is an essential component of any vaccine, and vaccine development strategies commonly involve the VP-1 G-H loop as a starting point. For example, novel vaccine approaches have been investigated which involve incorporation of the VP1 G-H loop sequence either alone, with other peptides or integrated on to other immunoparticles or virus vectors (Bachrach., (1975) as above, Kleid et al., (1981) Science. 214. 1125-1129, Strohmaier et al., (1982) as above, Laporte et al., (1973) as above, Volpina et al., (1999) Vaccine. 6. 577-584, Fischer et al., (2003) Journal of Virology. 13, 7486-7491, Bittle et al., (1982) Nature. 298. 30-33 and Pfaff et al., (1982) The EMBO Journal. 1. 869-874).

### SUMMARY OF ASPECTS OF THE INVENTION

The present inventors have surprisingly found that, despite prejudice in the art that the VP1 G-H loop is immunodominant and essential for the generation of a protective immune response, a vaccine based on virus in which a large section of the G-H loop has been deleted immunologically shows comparable levels of protection to a vaccine based on virus with a complete G-H loop.

Such a vaccine offers the significant advantage that vaccination can be distinguished from infection in an individual, by investigation of evidence of an anti-G-H loop immune response in the subject. The associated diagnostic assay relies on the detection of an immune response to part of a structural protein, making it much more sensitive and robust than the NSP-response detection methods currently in use.

Moreover, removal of the VP1 loop can be achieved at a molecular level, and production of the “loop negative” vaccine preparation does not rely on purification techniques, resulting in increased confidence that the vaccine will not contain any “loop positive” virus, which may result in the generation of false positives.

Thus, in a first aspect, the present invention provides, a foot and mouth disease (FMD) vaccine comprising a vaccinating entity comprising or capable of expressing a foot and mouth disease virus (FMDV) VP1 polypeptide having a deletion of at least seven amino acids in the G-H loop such that the VP1 polypeptide lacks an RGD motif,

wherein the vaccine is substantially free from a vaccinating entity comprising or capable of expressing an FMDV VP1 polypeptide having a complete G-H loop.

The vaccine may consist essentially of a vaccinating entity comprising or capable of expressing a foot and mouth disease virus (FMDV) VP1 polypeptide having a deletion in the G-H loop.

The vaccinating entity may, for example, be an inactivated FMD virus comprising the modified VP1 sequence.

Alternatively, the vaccinating entity may, for example, be a DNA vaccine, comprising a nucleotide sequence capable of encoding the FMD proteins needed to produce an FMDV empty capsid particle.

The exact deletion will depend on the serotype/strain of FMD virus upon which the vaccine is based. For an example, a vaccine which comprises or is capable of expressing a serotype A VP1 polypeptide may have a deletion of residues 142-154 in the G-H loop.

The vaccine of the present invention may be used for preventing FMD in a FMD-susceptible subject, such as a bovine subject.

Hence, in a second aspect, the present invention also provides a method for preventing FMD in a subject, which comprises the step of administering an effective amount of a vaccine of the first aspect of the invention to the subject.

Since the vaccinating entity of the invention has a deletion in the VP-1 G-H loop, it is possible to distinguish between animals which have been exposed to the vaccine, and those which have been exposed to wild-type virus (for example, by infection). Thus the present invention also relates to the use of a vaccine of the first aspect of the invention as a marker vaccine.

In a third aspect, the present invention provides a method for distinguishing between

a) FMD infected, sub-clinically infected or previously infected subjects; and

b) subjects vaccinated with a vaccine according to the first aspect of the invention, which comprises the step of investigating the presence of a VP-1 polypeptide comprising a G-H loop, or evidence of an immune response against the VP-1 G-H loop in the subject;

the presence of a VP-1 polypeptide comprising a G-H loop, or evidence of an immune response against the VP-1 G-H loop in the subject, indicating that the subject is or has been infected with FMDV.

The present invention also relates to a diagnostic test associated with the vaccine of the first aspect of the invention, i.e. a diagnostic test capable of detecting intact VP-1 G-H loops and/or a VP-1 G-H-loop specific immune response. Such a diagnostic test will be able to discriminate between vaccinated and infected individuals.

Thus in a fourth aspect, the present invention provides a differentiation assay to distinguish between

a) FMD infected, sub-clinically infected or previously infected subjects; and

b) subjects vaccinated with a vaccine according to the first aspect of the invention, which comprises a detection system for a VP-1 GH loop or for an anti-VP-1 G-H loop immune response.

The detection system may comprise an entity capable of binding specifically to the VP-1 G-H loop, such as a GH-loop specific antibody or an αvβ6 integrin. Alternatively, or in addition, the detection system may comprise an entity capable of detecting an anti-G-H loop immune response, such as an entity capable of detecting the presence of anti G-H loop specific antibodies.

Conveniently, the vaccine of the first aspect of the invention and its associated diagnostic test may be provided together, for example in the form of a kit.

### DESCRIPTION OF THE FIGURES

FIG. 1—A table showing amino acid substitutions and deletions between A+ and A− in VP1. Unique differences are highlighted by shading.

FIG. 2—Predicted structure of the VP 1 polypeptide of A+ and A− based on the co-ordinates of A10/Arg/61 (accession 1ZBE) using ESyPred3D.

a) A+; b) A−. Dotted box represents an approximation of the structure of the disordered residues of the A+VP 1 G-H loop based on known FMDV VP 1 G-H loop structures.

FIG. 3—Homologous virus neutralising antibody responses of cattle serum following vaccination.

Cattle were either vaccinated with A−(▴) or A+(▪). VNT: virus neutralising titre. Error bars represent 1 standard error (SE) above and below the mean.

FIG. 4—Day 21 virus neutralisation titres expressed as the log 10 reciprocal antibody dilution required for 50% neutralisation of 100 tissue culture infectious units. a) BEI-inactivated virus, 15 μg/2 ml dose, b) VNT titres. Means were calculated from unrounded data. Titre values presented in this table are mean values from two repeat tests.

FIG. 5—R values derived from day 21 virus neutralising titres displayed in table 6.4. Shaded values indicate those animals which would be theoretically protected from challenge with either A+ or A−. R values >0.3 were considered to indicate protection. a BEI-inactivated virus, 15 μg/2 ml dose.

FIG. 6—Mean serological anti-FMDV IgG Immunoglobulin's in cattle on day 21 post vaccination. Antibody titres are expressed as the reciprocal of the highest serum dilution with an OD value at least two times that of the serum samples at 0 day. Titres were converted into Log 10. Inactivated antigen concentration was 1 μg/ml. Titre values presented in this table are mean values from two repeat tests.

FIG. 7—R values derived from day 21 IgG immunoglobulin titres displayed in table 6.6. Shaded values indicate those animals which would be theoretically protected from challenge with either A+ or A−. R values >0.3 were considered to indicate protection. a BEI-inactivated virus, 15 μg/2 ml dose.

FIG. 8—Individual day 21 cattle serum titres expressed as the log 10 reciprocal antibody dilution derived from the LPBE. Titre values presented in this table are mean values from two repeat tests.

FIG. 9—R values derived from day 21 LPBE serum titres displayed in table 6.8. Shaded values indicate those animals which would be theoretically protected from challenge with either A+ or A−. R values >0.4 were considered to indicate protection. a BEI-inactivated virus, 15 μg/2 ml dose.

FIG. 10—Day 21 virus neutralisation titres and LPBE serum titres expressed as the log 10 reciprocal antibody dilution required for 50% neutralisation of 100 tissue culture infectious units. Titre values presented in this table are mean values from two repeat tests.

FIG. 11—Virus neutralisation titre ‘r’ values for FMDV type A field isolates. R values were determined from the mean titre from two independent tests. The r value is a measure of the relationship between virus strains and is given by r=reciprocal log of serum titre against heterologous/serum titre against the homologous. Any r value to the right of the red line would be considered as a protective level.

FIG. 12—Liquid Phase Blocking Elisa (LPBE) ‘r’ values for FMDV type A field isolates. R values were determined from the mean titre from two independent tests. The r value is a measure of the relationship between virus strains and is given by r=reciprocal log of serum titre against heterologous/serum titre against the homologous calculated from individual homologous control values on each plate and not calculated from homologous values presented in table 6.10. Any r value to the right of the red line would be considered as a protective level.

FIG. 13—Overview of diagnostic assay

FIG. 14—Day 21 serum from A+(RZ60 and 61) and A− (RZ65 and 67) vaccinated cattle (shown as duplicate wells). Serum was pre-absorbed with FMDV A− antigen (1 μg/ml) prior to reaction with ELISA plate captured A+ antigen (1 μg/ml)

FIG. 15—Sequence alignment of examples from seven FMDV serotypes showing a 13 amino acid deletion in the VP1 G-H loop.

FIG. 16—Important amino acid positions identified by Mabs including those in the VP1 G-H loop region of serotypes Asia, O, A and C1.

FIG. 17—Schematic models of various FMDV serotypes, showing deletions in the VP-1 G-H loop. A) Serotype A, 13 amino acid deletion; B) Serotype A, 21 amino acid deletion; C) Serotype C, 13 amino acid deletion; D) Serotype O, 13 amino acid deletion; E Serotype SA 1, 13 amino acid deletion); F) Serotype SAT 1, 13 amino acid deletion; G) Serotype SAT 2, 13 amino acid deletion; and H) Serotype SAT 3, 13 amino acid deletion.

### DETAILED DESCRIPTION

### Foot and Mouth Disease (FMD)

FMD is a highly contagious and economically devastating disease of animals, affecting domesticated ruminants, pigs and a large number of wildlife species (Alexandersen et al., (2003) as above) of which the causal agent is Foot-and-Mouth Disease Virus (FMDV). The disease is characterised by high fever for two or three days followed by the formation of blisters or lesions inside the mouth, on the mammary glands of females and also on the feet. The vesicles generally rupture within 1-2 days resulting in the formation of sore open wounds which if located on the feet cause lameness. Frequently, the healing of lesions is delayed by secondary bacterial infection of the wounds. Though most animals eventually recover from FMD, the disease can lead to myocarditis and death, especially in newborn animals. The long-term welfare of survivors can be poor, with many suffering secondary consequences such as mastitis, endometritis, chronic lameness and a substantial drop in milk yield (The Royal Society Report, (2002) as above).

The virus is present in secretions such as faeces, saliva, milk and breath and can infect susceptible animals through inhalation, ingestion, skin trauma and contact with mucosal membranes. Cattle, sheep and goats predominantly contract disease via the respiratory tract, whereas pigs are considerably less susceptible to aerosol infections requiring up to 600 times more tissue culture infectious doses (TCID50) of virus to become infected and therefore generally contract disease through ingestion (Donaldson and Alexandersen, (2002) Revue Scientifique et Technique Office International des Epizooties. 21. 569-575).

Following infection, the incubation period between infection and the appearance of clinical signs ranges from two to eight days but in some cases has been reported to be as long as 14 days (Alexandersen et al., (2003) as above). The severity of clinical signs is related to infectious dose, species, the level of immunity and the virus strain. It is sometime difficult to differentiate FMD clinically from other vesicular diseases, such as swine vesicular disease, vesicular stomatitis and vesicular exanthema. Laboratory diagnosis of any suspected FMD case is therefore usually necessary. The demonstration of specific antibodies to FMDV structural proteins in non-vaccinated animals, where a vesicular condition is present, is considered sufficient for a positive diagnosis.

The preferred procedure for the detection of FMDV antigen and identification of viral serotype is the ELISA. The test recommended by the World Organisation of Animal health is an indirect sandwich test in which different rows in multiwell plates are coated with rabbit antisera to each of the seven serotypes of FMD virus. These are the ‘capture’ sera. Test sample suspensions are added to each of the rows, and appropriate controls are also included. Guinea-pig antisera to each of the serotypes of FMD virus are added next, followed by rabbit anti-guinea-pig serum conjugated to an enzyme. A colour reaction on the addition of enzyme substrate, in the presence of a chromogen, indicates a positive reaction.

Alternatively, it is possible to use nucleic acid recognition methods to detect foot and mouth disease. Reverse transcription polymerase chain reaction (RT-PCR) can be used to amplify genome fragments of FMDV in diagnostic materials including epithelium, milk, and serum. RT combined with real-time PCR has a sensitivity comparable to that of virus isolation. Specific primers can be designed to distinguish between FMDV serotypes.

### Foot and Mouth Disease Virus (FMDV)

Foot and mouth disease virus (FMDV) is a positive sense, single stranded RNA virus and is the type species of the Aphthovirus genus of the Picornaviridae family. The virus is packaged in an icosahedral symmetric protein shell or capsid, approximately 28-30 nm in diameter. The capsid is composed of 60 copies each of four viral structural proteins, VP1, VP2, VP3 and the internally located VP4. VP1, 2 and 3 have similar tertiary structures containing a highly conserved β-barrel core. (Acharya et al., (1989) Nature. 337. 709-716). The FMDV RNA genome consists of an open reading frame encoding the four structural proteins, and at least eight non-structural proteins (NSP) (Leader, 2A, 2B, 2C, 3A, 3B, 3 Cpro, 3Dpol).

FMDV exists as seven antigenically distinct serotypes, namely O, A, C, SAT-1, SAT-2, SAT-3, and Asia-1, with numerous subtypes within each serotype. These serotypes show some regionality, and the O serotype is most common.

FMDV multiplication occurs in the cytoplasm of the host cell. The virus enters the cell through a specific cell surface receptor. FMDV favours the use of a class of receptors known as integrins for cell entry, but when the virus is tissue culture adapted it has been found to adapt to use an alternative receptor class to infect cells (Baranoski et al., (2000) Journal of Virology. 74. 1641-1647 and Baxt and Bachrach, (1980) Virology. 104. 391-405). FMDV can make use of integrins due to the presence of a conserved Arg-Gly-Asp motif located on VP1 G-H loop, as discussed below.

### Vaccine

The term ‘vaccine’ as used herein refers to a preparation which, when administered to a subject, induces or stimulates a protective immune response. A vaccine can render an organism immune to a particular disease, in the present case FMD.

The vaccine may be used propylactically, to block or reduce the likelihood of FMDV infection and/or prevent or reduce the likelihood of contracting FMD.

A vaccine comprises one or more vaccinating entity(ies) and optionally one or more adjuvants, excipients, carriers and diluents.

The vaccine may also comprise, or be capable of expressing, another active agent, for example one which may stimulate early protection prior to the vaccinating entity-induced adaptive immune response. The agent may be an antiviral agent, such as type I interferon. Alternatively, or in addition, the agent may be granulocyte-macrophage colony-stimulating factor (GM-CSF).

The vaccine may also comprise, or be capable of expressing, the FMDV non-structural protein 3D as a separate entity. The 3D protein has been shown to stimulate a strong humoral and cellular immune response in the host.

### Vaccinating Entity

The term ‘vaccinating entity’ as used herein is used to refer to the active component or an agent capable of producing (for example encoding) the active component of a vaccine. The active component is the entity which triggers an adaptive anti-FMDV immune response. Upon administration to a subject the presence of the active component stimulates antibody production or cellular immunity against FMDV.

In a first embodiment, the vaccinating entity of the present invention may be an inactivated, dead or attenuated form of FMDV.

The term ‘inactivated’ is used to describe a virus which has effectively lost the ability to replicate and cause infection.

Current commercially available FMD vaccines commonly contain chemically inactivated FMDV as the vaccinating entity. The virus may be inactivated by, for example, treatment with aziridines such as binary ethyleneimine (BEI). The virus used is usually a seed virus strain derived from cell culture, which, once inactivated, is then blended with suitable adjuvant/s and excipients. Two categories of chemically inactivated vaccine are currently available, namely water based and oil based vaccines (either single, double or complex oil emulsions). Water based vaccines, which are normally adjuvanted with aluminium hydroxide and saponin, are used for cattle, sheep and goats, whereas oil based vaccines, which induce more versatile and longer lasting immunity, can be used for all target species, including pigs.

A vaccine of the present invention, comprising inactivated FMDV having a modified VP1 polypeptide, can be produced in the same way as traditional vaccines. It can be produced in the same production plants as previously used for traditional vaccines, involving minimal change in production technology or technique.

In a second embodiment of the present invention, the vaccinating entity may be an antigenic portion of FMDV, which comprises the VP1 polypeptide. Vaccines have previously been developed comprising VP-1 alone, or in combination with other structural proteins.

Vaccines for FMD have also been developed which only contain the portions of the viral genome required for virus capsid assembly and lack the coding region for most of the viral nonstructural (NS) proteins (Grubman (2005) Biologicals 33:227-234). These “empty capsid” particles comprise the protein shell of a virus made up of the four protein subunits VP 1-4. As the empty capsid particles do not contain an FMDV RNA genome, they are non-infectious, but they mimic infectious virus antigenically. Some reports indicate that FMDV empty capsid particles are capable of inducing antibody responses at a similar level to that induced by the whole virus (Rowlands et al., (1975) J Gen Virol. 26. 227-38, Grubman et al., (1985) J Virol. 56. 120-6 and Francis et al., (1985) J Gen Virol. 66. 2347-54).

In a third embodiment, the vaccinating entity may be capable of expressing an FMDV VP1 polypeptide, having a deletion in the G-H loop. For example, the vaccinating entity may be or comprise a nucleotide sequence capable of expressing the modified VP1 polypeptide. The vaccinating entity may be or comprise a nucleotide sequence capable of expressing the capsid with a modified VP1 polypeptide.

The nucleotide sequence may be a RNA or DNA sequence. For example, the nucleotide sequence may be a synthetic RNA/DNA sequence, a recombinant RNA/DNA sequence (i.e. prepared by use of recombinant DNA techniques), a cDNA sequence or a partial genomic DNA sequence. The term “DNA vaccine” used herein refer to a vaccine comprising any type of nucleotide sequence as the vaccinating entity.

The vaccine may comprise a delivery system, capable of delivering the nucleotide sequence(s) to a host cell in vivo. The delivery system can be a non-viral or a viral delivery system.

Methods of non-viral gene delivery include using physical (carrier-free gene delivery) and chemical approaches (synthetic vector-based gene delivery). Physical approaches, including needle injection, electroporation, gene gun, ultrasound, and hydrodynamic delivery, employ a physical force that permeates the cell membrane and facilitates intracellular gene transfer. The chemical approaches use synthetic or naturally occurring compounds as carriers to deliver the nucleotide sequence into cells.

Non-viral gene delivery can conveniently be achieved by simple injection of, for example a plasmid, intradermally or intramuscularly into the subject.

Viral vector or viral delivery systems include but are not limited to adenoviral vectors, adeno-associated viral (AAV) vectors, herpes viral vectors, retroviral vectors (including lentiviral vectors) and baculoviral vectors.

The delivery system may comprise all or part of the FMDV genome, provided that it is capable of encoding the modified VP1 protein. For example, the delivery system may comprise an “empty capsid” gene cassette, such as the P1-2A cassette from FMDV O₁ Kaufbeuren. The vaccine may also comprise a gene capable of encoding the non-structural protein 3C (to cleave the capsid proteins). The vaccine may alternatively or in addition also comprise a gene capable of encoding the non-structural protein 3D as it is highly immunogenic and gives added T-cell stimulation.

DNA and subunit vaccines for FMD are advantageous over traditional inactivated whole-pathogen vaccines because that do not require high-security containment facilities for manufacture, thus alleviating any risk of virus “escape”. DNA and subunit vaccines are also non-infectious, easy to manipulate and prepare, inexpensive and are stable at room temperature, reducing the need for cold chain requirements.

DNA vaccination also has a number of advantages over protein-based vaccines. For example, a DNA vaccine results in endogenous expression of the antigen in vivo, allowing antigenic peptides to be presented to the immune system via both the MHC class I and II pathways, thereby priming not only CD4+ T-cells, but also CD8+ T-cells. DNA vaccines are thus able to induce both humoral and a strong cellular immune response. The use of plasmid DNA as a vaccine can also trigger the innate immune system of the host, through the unmethylated CpG motifs in the bacterial plasmid backbone and the Toll-like receptor 9 (TLR9).

The vaccine may lack the non-structural protein coding genes 2B and 2C which may interfere with cellular immune responses by down-regulating MHC and cytokine secretion (Moffat et al., (2005) J Virol. 79. 4382-95 and Moffat et al., (2007) J Virol. 81. 1129-39).

Many commercially available FMD vaccines are multivalent to provide cover against the different FMD serotypes. By the same token, the vaccine of the present invention may comprise a plurality of vaccinating entities, each directed at a different serotype and/or different subtypes within a given serotype. Each vaccinating entity comprises a deletion in the VP1 G-H loop.

The vaccine of the invention is substantially free from a vaccinating entity comprising or capable of expressing an FMDV VP1 polypeptide having a complete G-H loop. For example, the vaccine is substantially free from an inactivated virus preparation, subunit vaccine comprising or DNA vaccine encoding a wild-type VP1 polypeptide.

The term ‘substantially free’ as used herein means that the amount of a vaccinating entity comprising or capable of expressing an FMDV VP1 polypeptide having a complete G-H loop is such that, when the vaccine is administered to an animal, no detectable immune response is generated to the VP-1G-H loop. This means that the vaccine of the present invention can be used as a marker vaccine, as detection of an anti-VP-1 G-H loop immune response is then attributable to infection of the animal with wild-type FMDV.

The vaccine may consist essentially of one or more vaccinating entities comprising or capable of expressing a FMDV polypeptide having a deletion in the G-H loop. Such a vaccine would lack any other type of vaccinating entity, capable of inducing an anti-FMDV immune response. It may, however comprise other ingredients such as adjuvants, carriers, excipient or diluents which do not cause an adaptive immune response.

The vaccine may, for example, consist of a single vaccinating entity comprising or capable of expressing a FMDV polypeptide having a deletion in the G-H loop. Such a vaccine would lack any other vaccinating entity, capable of inducing an anti-FMDV immune response.

### VP1

VP1 is one of four viral structural proteins encoded for by the FMDV RNA, which form part of the viral capsid. The other structural proteins are known as VP2, VP3 and VP4, as discussed above.

Early work showed that, out of all the FMDV capsid proteins, only VP1 was capable of inducing a neutralising antibody response (Laporte et al., (1973) as above, and Bachrach et al., (1975) as above, although referred to as VP3 at that time).

The VP-1 G-H loop is a highly mobile loop located between B strands G and H of the FMDV structural protein VP1. The loop protrudes from the protein and is therefore highly exposed on the surface of the virus. The G-H loop contains a conserved Arg-Gly-Asp (RGD) motif which is known to interact with integrins and through this binding enable the virus to enter cells. Apart from this motif, the VP1 G-H loop is one of the most variable regions of the virus in terms of sequence homology.

The vaccine of the present invention comprises, or is capable of expressing, a VP1 polypeptide having a deletion in the G-H loop.

The term “polypeptide” is used in the conventional sense to mean a compound containing a plurality of amino acids, each linked by a peptide bond. The term “residues” is used to refer to amino acids in a polypeptide sequence.

The term “deletion” as used herein refers to total or partial removal of the VP1 G-H loop. The deletion may involve removal of seven or more amino acid residues from the G-H loop, including the RGD motif. For example, the deletion may involve removal of between 7 and 30 amino acids, 8 and 25, 9 and 20 amino acids. The deletion may involve deletion of about 13, for example between 11 and 15 or 12 and 14 amino acids.

The VP1 loop is found in the section from amino acid 129 to amino acid 172 of the VP1 polypeptide. The deletion may involve removal of all or a part of this 30 amino acid section, providing that the deletion is at least 7 amino acids in length.

The minimum deletion is therefore the RGD motif+4 amino acids, in any orientation (i.e. RGDXXXX, XRGDXXX . . . XXXXRGD). Based on studies which have looked at another picornavirus ((HRV2). P. V. Barnett et al (1995) Journal of General Virology. 76. 1255-1261) the maximum deletion may be RGD+18 amino acids, in any orientation provided that the deletion is within the region between βG2 and βH loops.

Preferably, therefore, the deletion involves removal of between 7 and 21 amino acids from the G-H loop, including the RGD group.

The deletion involves removal of a section of the VP1 loop, such that the two ends are joined forming either a shortened VP1 loop, in the case of a partial deletion; or complete removal of the VP1 loop, in the case of a total deletion. Preferably, the deletion is simply a removal of amino acids and does not involve replacement of any deleted amino acids with alternatives (i.e. a substitution).

Apart from the deletion of the VP1 loop, the remainder of the VP1 polypeptide preferably has a high degree of identity with the wild-type VP1 polypeptide. This will maximise the number of antigenic sites remaining on the molecule for the generation of an anti-FMDV immune response. The modified VP1 polypeptide (excluding the VP1 loop section) may for example have, 80, 90, 95 or 99% identity with the wild-type VP1 polypeptide of the appropriate serotype.

Since the VP1 G-H loop is variable between FMDV serotypes, the nature of the deletion ranges will vary. In the context of the present invention, the VP1 G-H loop in any serotype is defined as the region between beta sheets βG2 and βH. Using O₁ BFS as an example, this region starts VYN (residues 129, 130, 131) and ends NYG (residues 164, 165, 166) (Table 1). Examples from all seven serotypes have been aligned to O₁ BFS 1860 to give the relative start and end of the VP1 G-H loop (also shown in Table 1).

TABLE 1
βG-βH loop position in each FMDV serotype defined by alignment
with the structure of O₁ BFS 1860.

| | βG-βH | βG-βH | |
|---|---|---|---|
| Serotype | start | end | Length |
| O | 129 | 166 | 38 |
| | 130 | 167 | 38 |
| A | 129 | 164 | 36 |
| | 129 | 165 | 37 |
| C | 129 | 162 | 34 |
| | 130 | 163 | 34 |
| | 130 | 164 | 35 |
| Asia 1 | 128 | 163 | 36 |
| | 129 | 164 | 36 |
| | 129 | 165 | 37 |
| SAT 1 | 130 | 172 | 43 |
| SAT 2 | 129 | 168 | 40 |
| SAT 3 | 128 | 170 | 43 |
| | 129 | 171 | 43 |

### Serotype A

As shown in Table 1, the G-H loop of the VP1 polypeptide of wild-type FMDV serotype A spans amino acids 129-164 or 129-165 depending on strain.

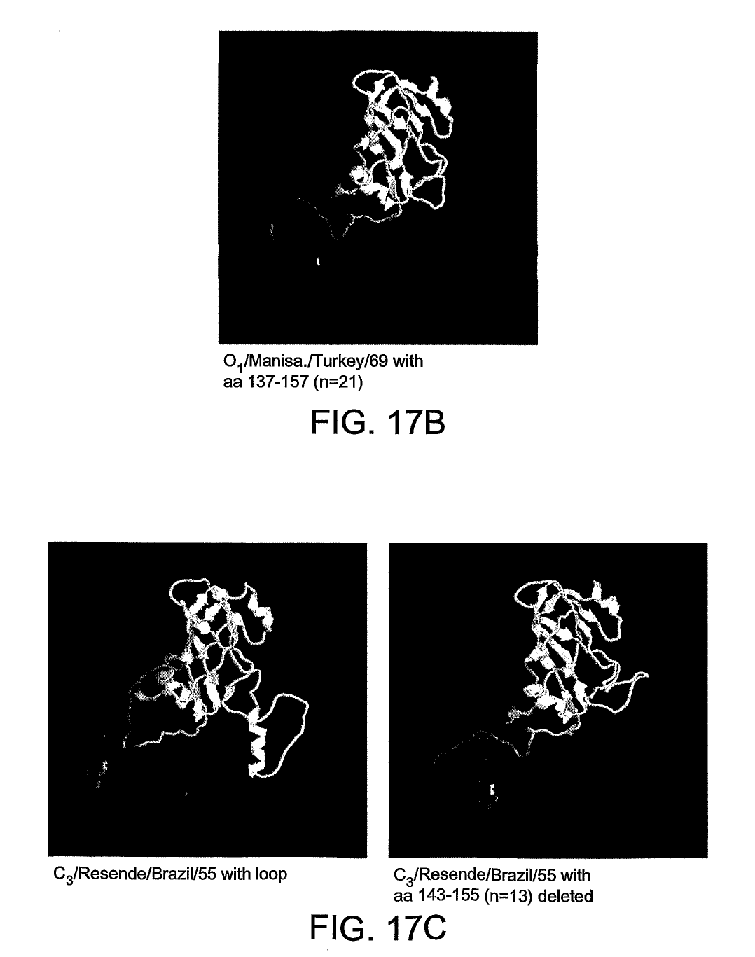

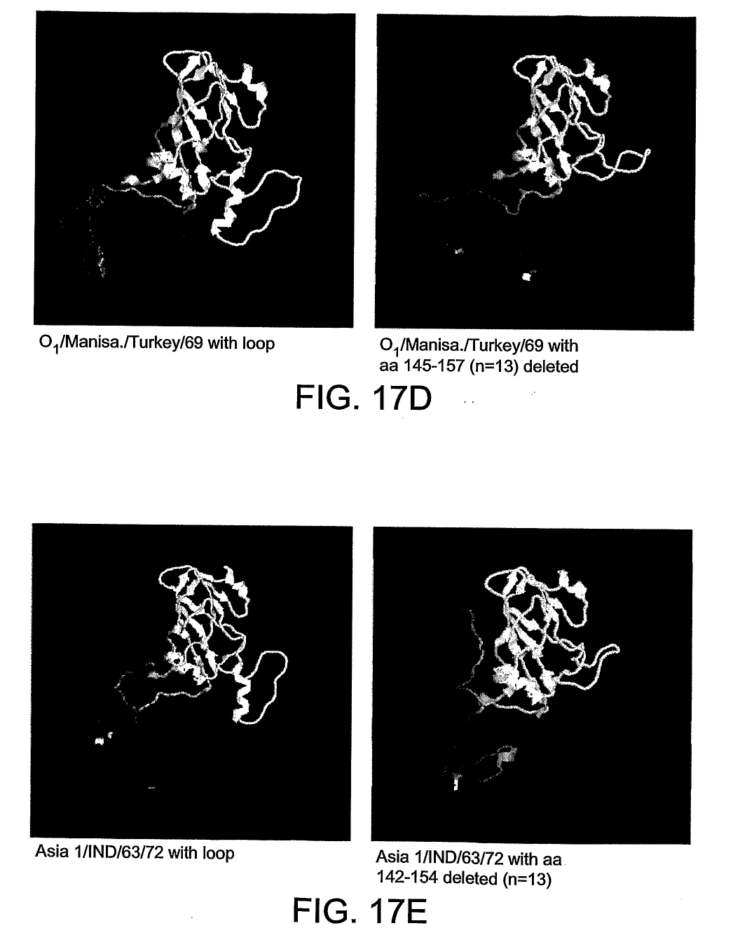

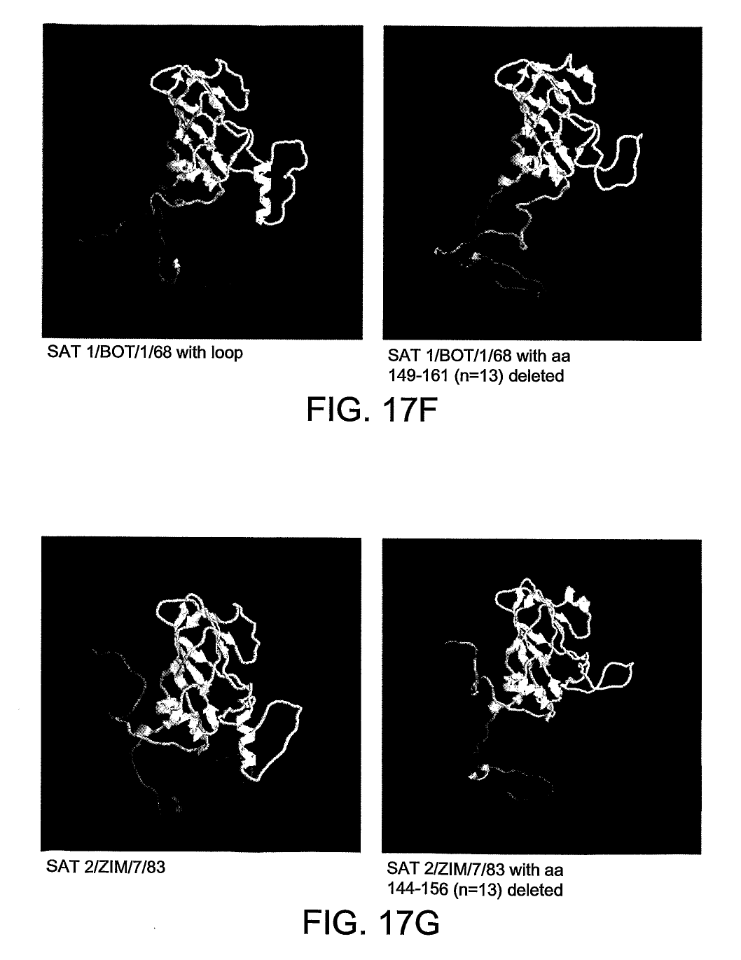

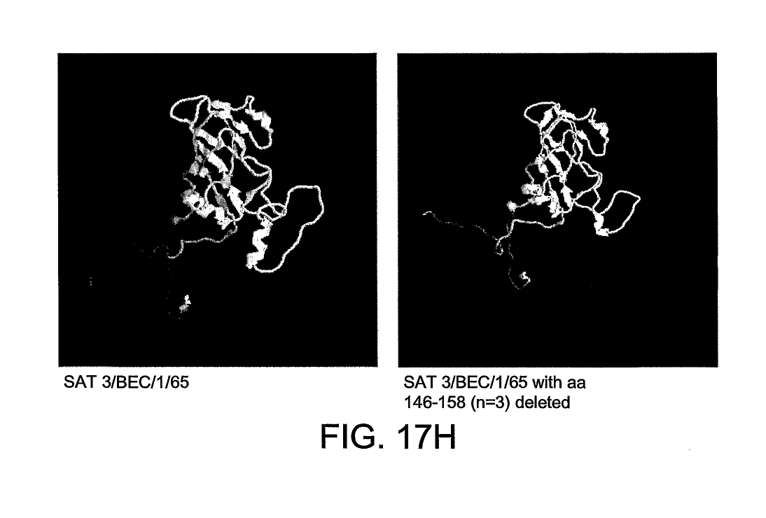

A modified polypeptide having a deletion of the section spanning residues 140-153 is specifically exemplified in the Examples section and modelled in FIG. 17A.

It is predicted, however, that shorter and longer deletions, and deletions transposed in a 5′ or 3′ direction will also be effective. FIG. 17 B shows a model of a 21 amino acids deletion: (O₁/Manisa./Turkey/69 with aa 137-157 (n=21), which is also predicted to be effective.

### Serotype C

As shown in Table 1, the G-H loop of the VP1 polypeptide of wild-type FMDV serotype C spans amino acids 129-162 or 130-163 or 130-164 depending on strain.

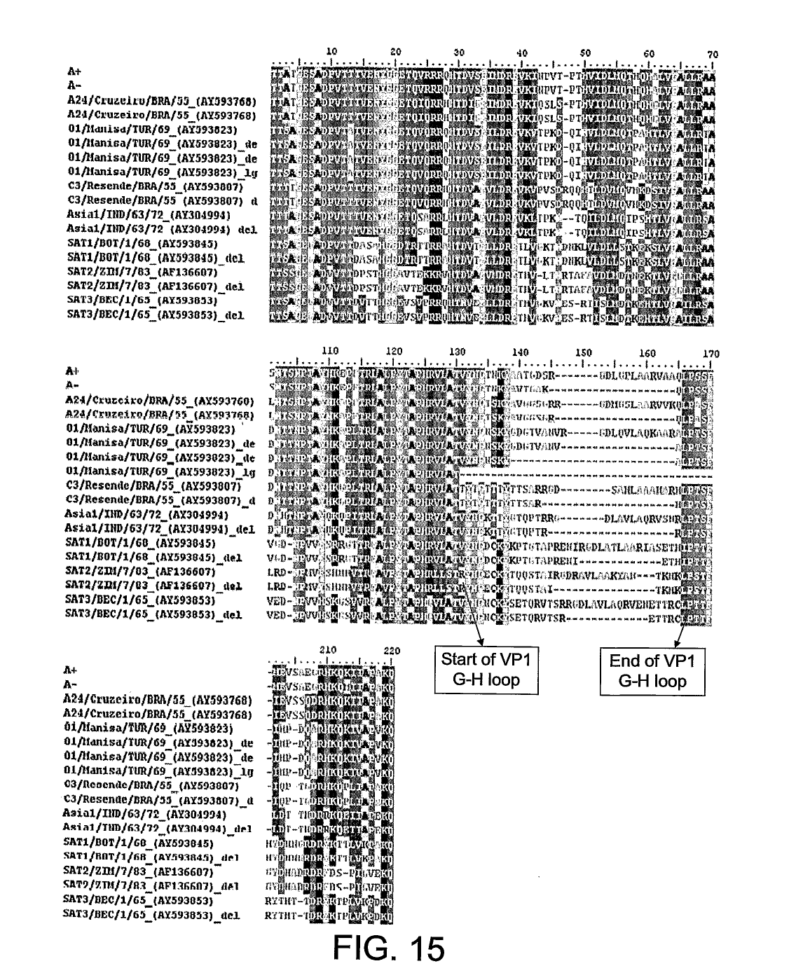

A 13 amino acid deletion, equivalent to the one described above for serotype A, would span residues the same residue numbers when aligned (FIG. 15). FIG. 17C shows a model of serotype C showing such a deletion.

The G-H loop of VP1 includes the major antigenic site for the FMDV which, for serotype C has been termed antigenic site A (Mateu (1995) Virus Res 38:1-24). In addition to site A, two main neutralisation sites have been described for FMDV serotype C: the carboxyterminal region of VP1, termed site C (residues 200-205) and a discontinuous site termed site D (residues 193-197). The modified VP1 protein, having a deletion in the G-H loop, may comprise one or both of these sites.

### Serotype O

As shown in Table 1, the G-H loop of the VP1 polypeptide of wild-type FMDV serotype O spans amino acids 129-166 or 130-167 depending on strain.

A 13 amino acid deletion, equivalent to the one described above for serotype A, would span residues the same residue numbers when aligned (FIG. 15). FIG. 17D shows a model of serotype O showing such a deletion.

### Serotype Asia1

As shown in Table 1, the G-H loop of the VP1 polypeptide of wild-type FMDV serotype Asia 1 spans amino acids 129-163 or 129-164 or 129-165 depending on strain.

A 13 amino acid deletion, equivalent to the one described above for serotype A, would span residues the same residue numbers when aligned (FIG. 15). FIG. 17E shows a model of serotype Asia1 showing such a deletion.

### Serotype SAT-1

As shown in Table 1, the G-H loop of the VP1 polypeptide of wild-type FMDV serotype Sat 1 spans amino acids 130-172 depending on strain.

A 13 amino acid deletion, equivalent to the one described above for serotype A, would span residues the same residue numbers when aligned (FIG. 15). FIG. 17F shows a model of serotype SAT-1 showing such a deletion.

### Serotype SAT-2

As shown in Table 1, the G-H loop of the VP1 polypeptide of wild-type FMDV serotype SAT2 spans amino acids 129-168 depending on strain.

A 13 amino acid deletion, equivalent to the one described above for serotype A, would span residues the same residue numbers when aligned (FIG. 15). FIG. 17G shows a model of serotype SAT-2 showing such a deletion.

### Serotype SAT-3

As shown in Table 1, the G-H loop of the VP1 polypeptide of wild-type FMDV serotype SAT 3 spans amino acids 128-170 or 129-171 depending on strain.

A 13 amino acid deletion, equivalent to the one described above for serotype A, would span residues the same residue numbers when aligned (FIG. 15). FIG. 17H shows a model of serotype SAT-3 showing such a deletion.

A number of investigators have removed just the RGD motif, to investigate the infectivity of viruses lacking this cell binding domain, or substituted it and some of its flanking sequences with alternative sequences (Baranowski et al (2001) Virology 288 192-202; Frimman et al (2007) Vaccine 25: 6191-6200; U.S. Pat. No. 5,612,040). The present invention, however, represents for the first time that a vaccine having a deletion of seven or more amino acids from the G-H loop has been tested, and shown to induce an immune response in an FMD-susceptible animal which is predicted to be protective.

Moreover, removal of just the RGD motif would not be sufficient to prevent the generation of anti-VP-1 G-H loop antibodies, meaning that a vaccine based on such a virus would not be useful as a marker vaccine. In order to avoid the generation of anti-VP-1 G-H loop antibodies, it is necessary to remove the antigenic region which, it is predicted, would necessitate removal of at least 7 amino acids.

### Vaccination Method

The present invention also provides a method of preventing FMD in a subject by administration of an effective amount of a vaccine according to the first aspect of the invention.

The term ‘preventing’ is intended to refer to averting, delaying, impeding or hindering the contraction of FMD. The vaccine may, for example, prevent or reduce the likelihood of an infectious FMDV entering a cell.

The subject may be any animal which is susceptible to FMD infection. FMD susceptible animals include cattle, sheep, pigs, and goats among farm stock, as well as camelids (camels, llamas, alpacas, guanaco and vicuna). Some wild animals such as hedgehogs, coypu, and any wild cloven-footed animals such as deer and zoo animals including elephants can also contract FMD.

The subject vaccinated according to the present invention may be a cloven-hoofed animal. In particular, the vaccine of the present invention may be used to treat a bovine subject.

### Administration

Current FMD vaccines can be given as routine vaccinations or emergency doses.

For FMD vaccines currently on the market, in order to establish a satisfactory level of immunity using routine vaccination, the World Organisation for Animal health recommends a primary course consisting of two inoculations, 2-4 weeks apart, followed by revaccination every 4-12 months. Routine vaccination involves administration of a low payload dose, for example about 3PD₅₀. The exact frequency of revaccination will depend on the epidemiological situation and the type and quality of vaccine used.

Routine vaccination against FMD is used in many countries or zones recognised as ‘free from foot and mouth disease with vaccination’ and in countries where the disease is endemic. Many disease-free countries maintain the option to vaccinate and have their own strategic reserves of highly concentrated inactivated virus preparations. This strategy relies on preparing vaccines to known strains of the virus currently circulating and assuming any disease will be introduced from such a ‘hot spot’. These antigen reserves offer the potential of supplying formulated vaccine in an ‘emergency’ at short notice and were used to great effect in the Netherlands in 2001, thus preventing an outbreak on the same scale as seen in the UK.

For emergency vaccination, a higher payload dose is given of, for example 6PD₅₀ or greater, commonly given as a single IM inoculation.

Where the vaccine is a DNA vaccine it may be administered by methods known in the art. The most suitable delivery method will depend on the delivery system used to deliver the nucleotide sequence to a target cell. For example, for plasmid administration, the plasmid preparation may be administered intramuscularly, intradermally or a combination of the above.

A delivery system capable of expressing an adjuvant may be administered to the subject simultaneously, separately or sequentially.

The vaccine may be administered following a prime-boost regime. For example, the subject may be primed with a DNA vaccine and boosted with a protein vaccine. For example, the administration regime may involve one, two or three DNA empty capsid vaccinations at three week intervals, followed by a protein boost of the capsid containing the VP-1 loop deletion.

An advantage of such a prime-boost regime is that it has been shown that it can give rise to cross-serotype reactivity as well as a greatly enhanced immune response. Hence animals vaccinated with a prime boost strategy may have increased protection against a heterologous challenge (Li et al (2008) Vaccine in press).

### Marker Vaccine

The vaccine of the present invention may be used as a marker vaccine. A marker vaccine is one capable of distinguishing between a vaccinated and infected subject. The vaccine of the present invention acts as a marker vaccine because, in contrast to infection with wild-type FMDV, it will not generate an immune response to the G-H loop of the VP1 polypeptide.

As mentioned above, the G-H loop of VP1 is the immunodominant region, so the differentiation assay is very “clean” and has little background. In this respect, if an animal has been exposed to wild-type virus, it should produce a clearly detectable anti-G-H loop immune response, which would be absent in an animal exposed to a vaccine of the present invention.

This offers considerable advantages over the detection of non-structural proteins (NSPs), which is the cornerstone of current procedures for distinguishing between individuals vaccinated with conventional FMD vaccines and FMDV infected individuals. As explained in the background section, the antibody response to NSPs in infected individuals is highly variable and is commonly too low to detect in individual animals, necessitating herd-level diagnosis. Moreover, it is possible for the vaccine to be contaminated with NSPs, leading to high background in the differentiation assay.

The differentiation assay of the present invention is “cleaner” and has less background because structural proteins are present at higher concentrations than non-structural proteins, and because the VP1 G-H loop is highly immunogenic.

Having said that, the currently available NSP differentiation tests could be used in conjunction with the differentiation tests of the present invention, as a cross-check, in order to validate results.

### Differentiation Assay

The present invention also describes a method for distinguishing between

i) FMD infected, sub-clinically infected or previously infected subjects; and
ii) subjects vaccinated with a vaccine according to the present invention.

The term ‘infected’ describes the state of a subject which contains FMDV in the body, and in which symptoms of FMD disease may or may not be displayed.

‘Previously infected’ refers to subjects which were once infected and showed symptoms of FMD but have later recovered the disease, and FMDV is no longer detectable within the body.

The term ‘sub-clinically infected’ describes the state of a subject which contains FMDV, but no symptoms of disease caused by the pathogens are displayed. Persistence of FMD, that leads to the animal becoming an asymptomatic carrier, is defined as the ability to recover virus from oesophageal-pharyngeal fluid 28 days or more post infection (Kitching, (2002) as above).

The method of the present invention may be used to distinguish between vaccinated and infected subjects at an individual animal level. This is in contrast to the less sensitive NSP differentiation tests whose use is limited to herd-level diagnosis.

### Detection System

In order to distinguish between

i) FMD infected, sub-clinically infected or previously infected subjects; and
ii) subjects vaccinated with a vaccine according to the present invention an investigation is carried out to detect

a) the presence of VP-1 G-H loops in the subject, which indicated the presence of wild-type virus; and/or

b) evidence that an immune response has ever been generated to the VP1 G-H loop in the subject. If there is evidence that an anti-G-H loop has been generated in an animal, this indicates that the animal has previously been exposed to wild type virus (i.e. infected) rather than the vaccine.

Detection of intact VP-1 GH loop (i.e. detection of wild-type virus) may be used to identify an infected or subclinically infected animal which still harbours the virus. Detection of G-H loop may be achieved for example, using a protein which binds specifically to the G-H loop, such as an G-H loop specific antibody, or an αvβ6 integrin.

WO06/00740 describes suitable αvβ6 integrins for use in the present differentiation assay.

The detection of an anti G-H loop immune response may involve any of the parts of the adaptive immune response which can be specifically attributed to the G-H loop. This includes the generation of T-cells specific for G-H loop peptides, and the generation of G-H loop specific antibodies.

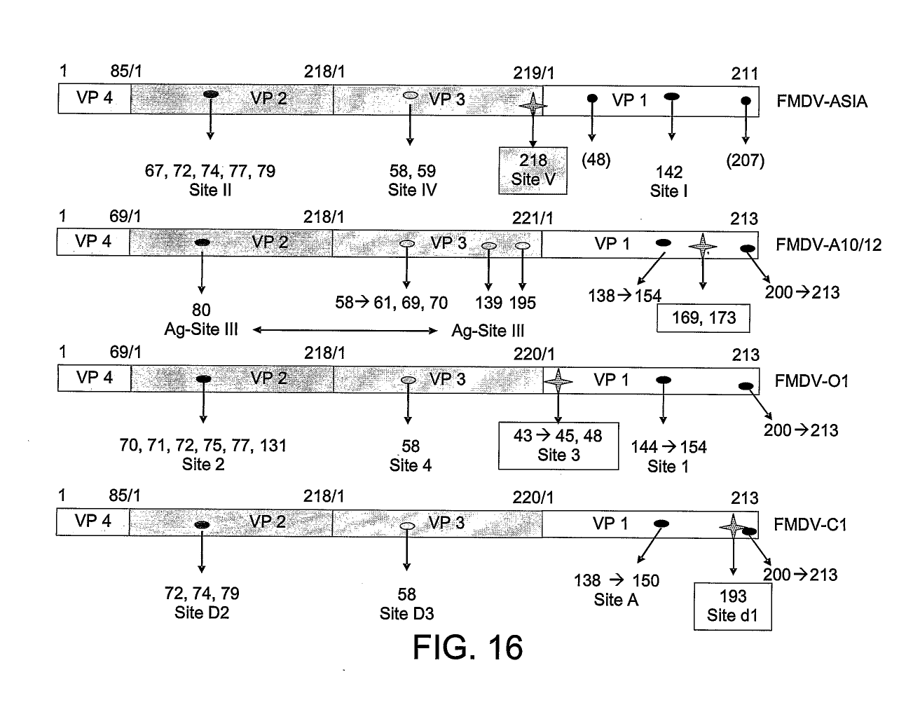

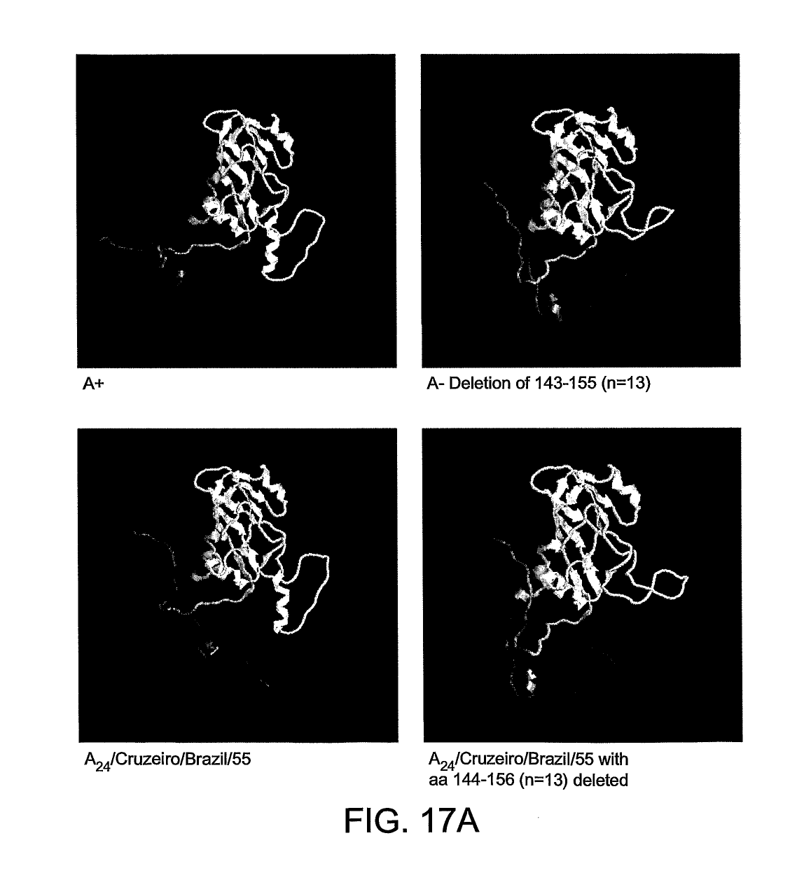

Conveniently, the detection system is capable of detecting one or more antibodies which binds to an epitope of the VP1 G-H loop. The epitope is fully or partially removed by the G-H loop deletion in the vaccine, such that the antibody does not recognise or bind specifically to the VP1 polypeptide of the vaccinating entity of the invention. Many VP-1 G-H loop specific antibodies have been developed and are known in the art. The critical amino acid positions identified by Mabs in various serotypes are shown in FIG. 16.

The antibody may be any class of immunoglobulin, including IgM, IgD, IgG, IgA and IgE.

The phrase “antibodies to the G-H loop” is intended to have the same meaning as “anti-G-H loop antibodies, in describing antibodies that bind specifically to the VP1 G-H loop, and do not significantly cross-react with other epitopes elsewhere on VP1 or the FMDV.

Detection of an anti G-H loop immune response may be carried out on a sample from the subject, such as a blood sample. In order to distinguish between a blood sample from a vaccinated subject, which will comprise antibodies to the vaccinating entity, but not G-H loop antibodies; and a sample from an infected subject, which will comprise antibodies to the FMDV, including G-H loop antibodies, it is convenient if the detection method includes a step where non-G-H loop antibodies are removed.

Removal of non-G-H loop antibodies can be accomplished by pre-absorbing the serum sample with an entity lacking the G-H loop, such as the vaccinating entity. The vaccinating entity can be immobilised by binding to e.g. a solid surface such as a plate or beads, and should be in an excess, to ensure that all non-G-H loop antibodies are absorbed. Any antibodies which do not bind to the vaccinating entity can therefore be assumed to be a G-H loop antibody. The presence of such antibodies can then be detected by method known in the art, such as ELISA. The term “removal” indicates that the non-G-H loop antibodies are bound or immobilised such that they do not substantially interfere with the G-H loop antibody detection step. The bound non-G-H loop antibodies may, however, still be within the serum sample. In other words, the non-G-H loop antibodies are effectively “removed” from the interaction, which may or may not involve physically removal.

The presence of G-H loop specific antibodies in a sample (before or after a pre-absorption step mentioned above) could also be detected using a G-H loop peptide in the absence of the remainder of the VP1 molecule. For example by using a fusion protein comprising the GH loop and a carrier protein.

The invention will now be further described by way of Examples, which are meant to serve to assist one of ordinary skill in the art in carrying out the invention and are not intended in any way to limit the scope of the invention.

### EXAMPLES

### Example 1

### Investigation of the Sequence and Predicted Structure of A− VP1

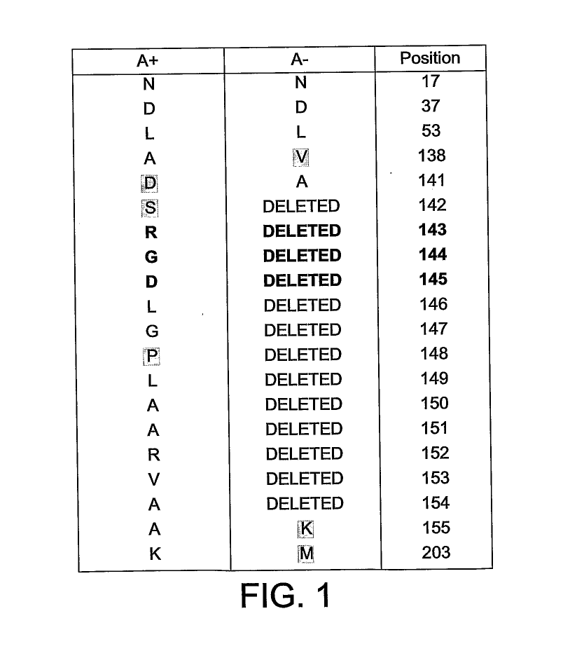

The VP1 coding region of viruses A− and A+ were amplified by PCR and sequenced and compared to each other to identify amino acid substitutions (FIG. 1), of which there are 4 (residues 138, 141, 155 and 203) and deletions, of which there are 13 in A− (residues 142-154). These deletions and substitutions observed within VP1 involve site 1 (residues 144, 148 and 154) and site 5 (residue 149) critical residues. Changes at residues 17, 37, 53 and 203 of VP1 have not been shown to be antigenically significant locations (Bolwell et al., (1989) Journal of General Virology. 70. 59-68, Thomas et al., (1988) Journal of Virology. 62, 2782-2789, Baxt et al., (1989) Journal of Virology. 63. 2143-2151 and Saiz et al., (1991) Journal of Virology. 65. 2518-2524). Preliminary analysis of amino acid differences for the rest of the capsid between A+ and A− revealed no other differences.

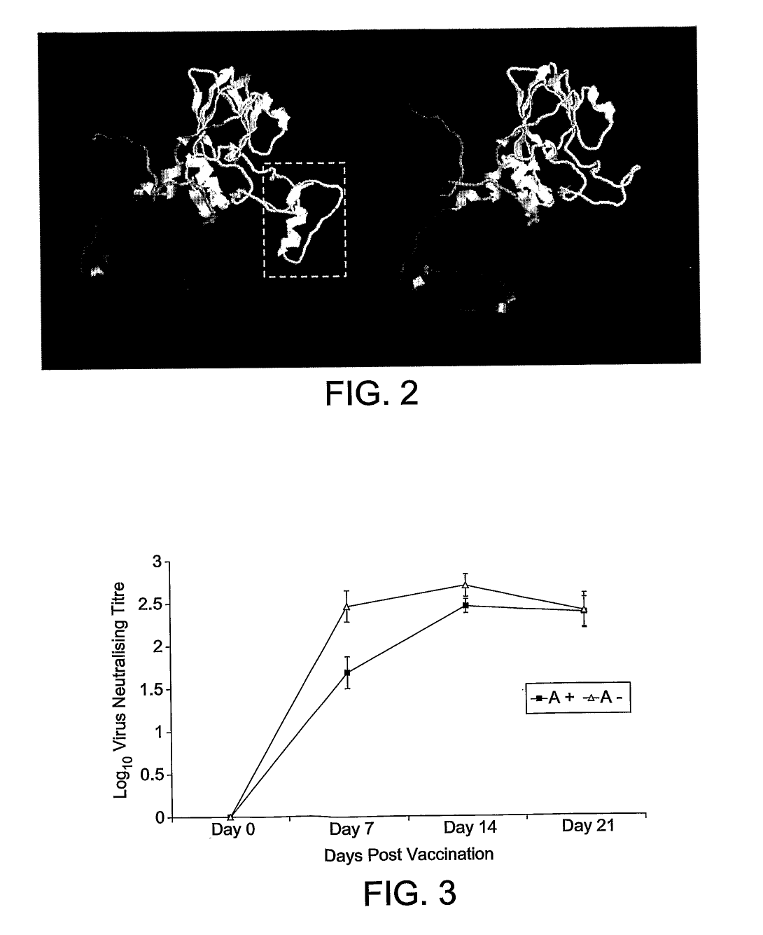

The VP1 coding sequences for A+ and A− were then used to predict the structure of the VP1 polypeptide of A+ and A−, based on the co-ordinates of A10/Arg/61 (database accession no. 1ZBE) (Fry et al., (2005) Journal of General Virology. 86. 1909-20) and using ESyPred3D (Lambert et al., (2002) Bioinformatics. 18. 1250-1256). This was done in order to determine whether the loss of the 13 amino acids had resulted in obvious structural changes elsewhere on VP1 which could have altered discontinuous epitopes. As shown in FIG. 2, the VP1 G-H loop deletion appears not to have influenced the surrounding VP1 structures, but simply joined the two ends into a shortened VP1 G-H loop. This is consistent with the view that serological variation observed between A+ and A− is likely to be due to VP1 G-H loop differences between the two viruses. This assumption is also supported by the fact that in A serotype the VP1 G-H loop is believed to have a loose association with the capsid and therefore it is usually recognised as a functionally independent site (Bolwell et al., (1989) as above).

### Example 2

### Comparison of the Potency of A+ and A− Vaccine Strains

Sera were collected on days 0, 7, 14 and 21 were tested by VNT to assess the virus neutralising antibody response to vaccination and to estimate the relative potency of the vaccine strains. FIG. 3 shows that vaccines prepared from A− or A+ produced a similar response and induced measurable levels of anti-FMDV neutralising antibody detectable as early as 7 days post vaccination. The animals which received the vaccine prepared from A− had a higher serum neutralising antibody titre on days 7 to 14 post vaccination than the animals vaccinated with A+. However by day 21 identical neutralising antibody titres were measured and since day 21 serum is used for all assays the vaccines were considered comparable in terms of potency.

### Example 3

### Comparison of the Anti-FMDV Serum Neutralising Antibody Response Between A+ and A− Strains by VNT

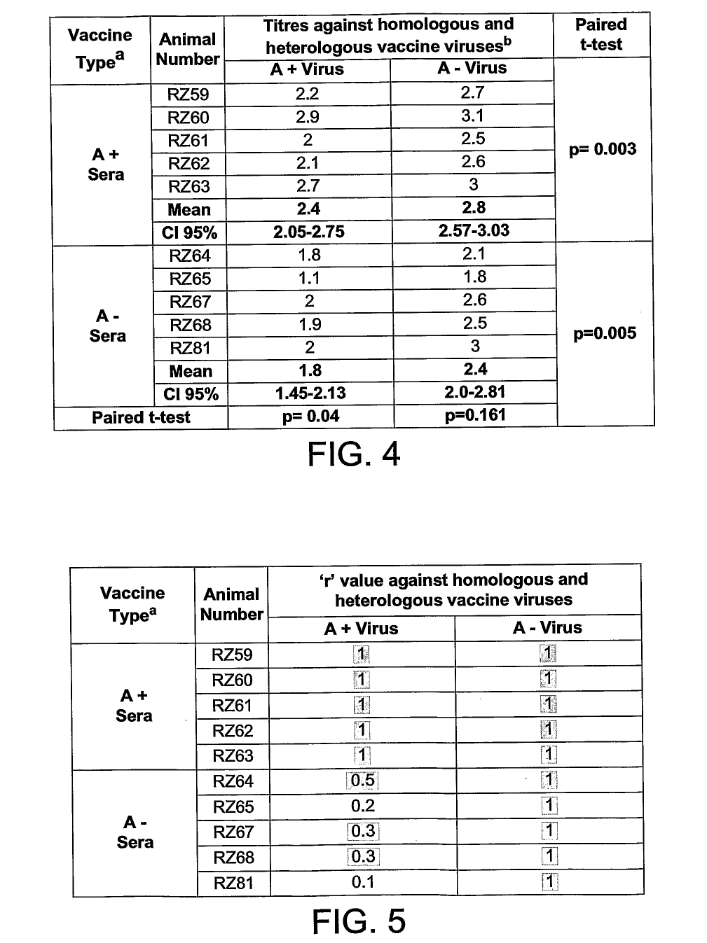

In order to measure any differences in the ability of sera raised against A+ or A− to neutralise virus, day 21 post vaccination serum neutralising antibodies were measured by virus neutralisation (VNT) (FIG. 4).

Data may be analysed by comparing the differences in reactivity of each antiserum against homologous and heterologous viruses (across row analysis) and the differences in reactivity of each virus against homologous and heterologous antisera (down column analysis) (FIG. 4). Serum titres can then be used to calculate r values which were subsequently used to predict the likelihood of an A− vaccine protecting cattle from challenge with a wild type virus such as A+.

The data shows that the homologous virus neutralising antibody titres were not significantly different (p=0.94). Across row analysis shows that A+ sera is significantly (p=0.003) more efficient at neutralising A− than A+ virus and that A− sera is significantly (p=0.005) more efficient at neutralising A− virus. Down column analysis shows that A+ sera is significantly (p=0.04) more efficient than A− sera at neutralising A+ virus and that although not significant (p=0.161), A+ sera is more efficient at neutralising A− virus than the homologous reaction. Commonalities which exist in the data sets include i) A+ sera has greater virus neutralisation capabilities than A− sera and ii) A− virus is more easily neutralised than A+ virus. In addition to this analysis, interpretation of the r values (FIG. 5) indicates that protection from challenge with A+ would be achieved in three out of the five animals vaccinated with A−.

### Example 4

### Comparison of IgG Titres Between A+ and A− Strains

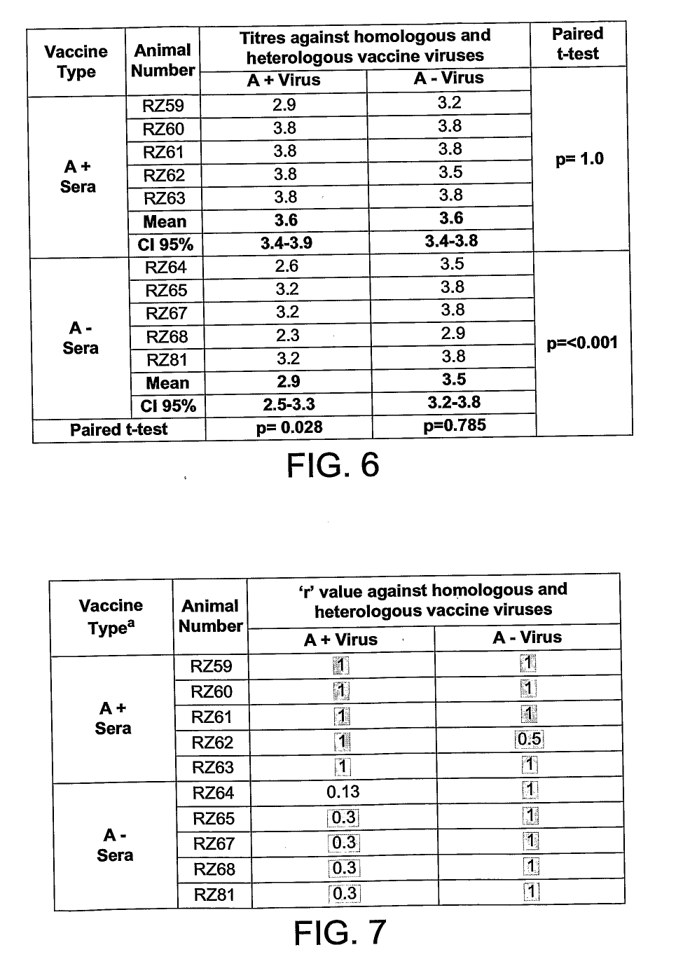

In order to measure any differences in the ability of antibody to bind to A+ or A− virus, day 21 post vaccination serum IgG titres were measured by indirect sandwich ELISA (FIG. 6). IgG titres were measured, firstly because they represent the predominant immunoglobulin subtype at this time point after vaccination, and secondly because the IgG isotype is involved in antibody-enhanced opsonisation and subsequent phagocytosis.

Data were analysed by comparing the differences in reactivity of each antiserum against homologous and heterologous viruses (across row analysis) and the differences in reactivity of each virus against homologous and heterologous antisera (down column analysis). Serum titres were then used to calculate r values (FIG. 7) which were subsequently used to predict the likelihood of an A− vaccine protecting cattle from challenge with a wild type virus such as A+.

For each serum, the mean homologous anti-FMDV IgG titres were not significantly different (p=0.82), differing by only 0.1 log 10 between cattle vaccinated with either A+ (3.6 log 10) or A− (3.5 log 10) (FIG. 6).

Across row analysis shows that there is no significant difference (p=1.0) in the ability of sera raised against A+ to bind to either A+ or A− inactivated antigen and that A− sera can bind to A− virus at significantly higher (p=<0.001) serum dilution than to A+ virus. Down column analysis shows that there is also a significant difference (p=0.028) between the ability of A+ and A− sera to bind to A+virus. A+ sera can bind to A+ virus at a higher dilution than A− sera, whereas there is no significant difference (p=0.785) in the ability of A+ and A− sera to bind to A− virus. Commonalities which exist in the data sets include i) A+ sera has greater binding capabilities to A+ virus than A− sera and ii) the binding of either A+ or A− serum IgG immunoglobulin's to A− virus was not compromised by the VP1 G-H loop deletion. In addition to this analysis, interpretation of the r values (FIG. 7) indicates that protection from challenge with A+ would be achieved in four out of the five animals vaccinated with A−.

### Example 5

### Comparison of the Anti-FMDV Serum Neutralising Antibody Response Between A+ and A− Strains by LPBE

Liquid phase blocking ELISA (LPBE) is another assay which can be used to determine the differences in the ability of A+ and A− sera to bind to A+ and A− virus. It differs from the assay used to detect the binding of IgG immunoglobulins in that it is a competition test and measures the ability of the test sera to block out the binding of a polyclonal guinea pig detection antibody.

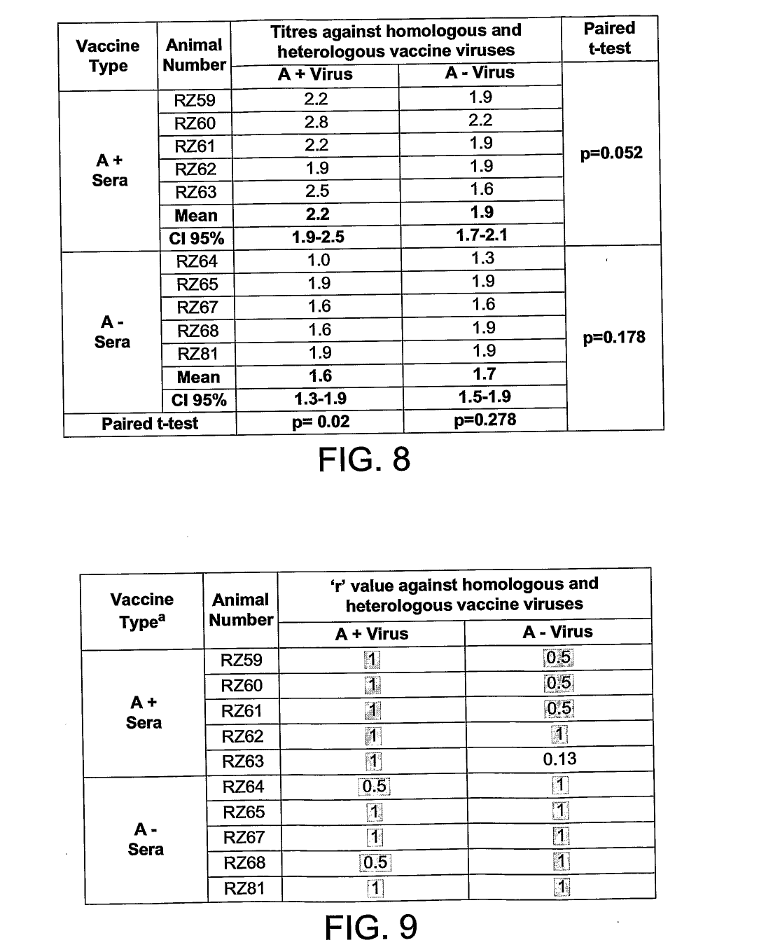

The data presented in FIG. 8 analysed by comparing the reactivity of each antiserum against homologous/heterologous viruses (across row analysis) and the differences in reactivity of each virus against homologous and heterologous antisera (down column analysis). Serum titres were then used to calculate r values (FIG. 9) which were subsequently used to predict the likelihood of an A− vaccine protecting cattle from challenge with a wild type virus such as A+.

In this case homologous titres were significantly different (p=0.018), with the A+ sera titre being 3 fold greater than the A− sera titre. Across row analysis shows that the A+ sera can block guinea pig polyclonal sera from binding to A+ at a much higher serum dilution (p=0.052) than its ability to block its binding to A− virus and that the homologous response is not significantly different (p=0.178) from the heterologous response for the A− sera. Down column analysis shows that the A+sera can block the binding of the guinea pig polyclonal detection antibody to A+virus at much higher serum dilutions than the A− sera and that as above there is no significant difference (p=0.278) between the ability of A+ and A− sera to block out binding of the guinea pig polyclonal sera binding to A−. Commonalities observed in the data are that the A+ sera can block out binding of the guinea pig detection antibody to A+ virus at much higher serum dilutions than A− sera. Results presented here also suggest that neither the A− nor A+ sera can bind to the region of the VP1 G-H loop which remains in A−, whereas the polyclonal guinea pig detection antibody can.

In addition to this analysis, interpretation of the r values (FIG. 9) indicates that protection from challenge with A+ would be achieved in five out of the five animals vaccinated with A−.

### Example 6

### Comparison of Potency of A+ and A− Against a Panel of Field Isolates

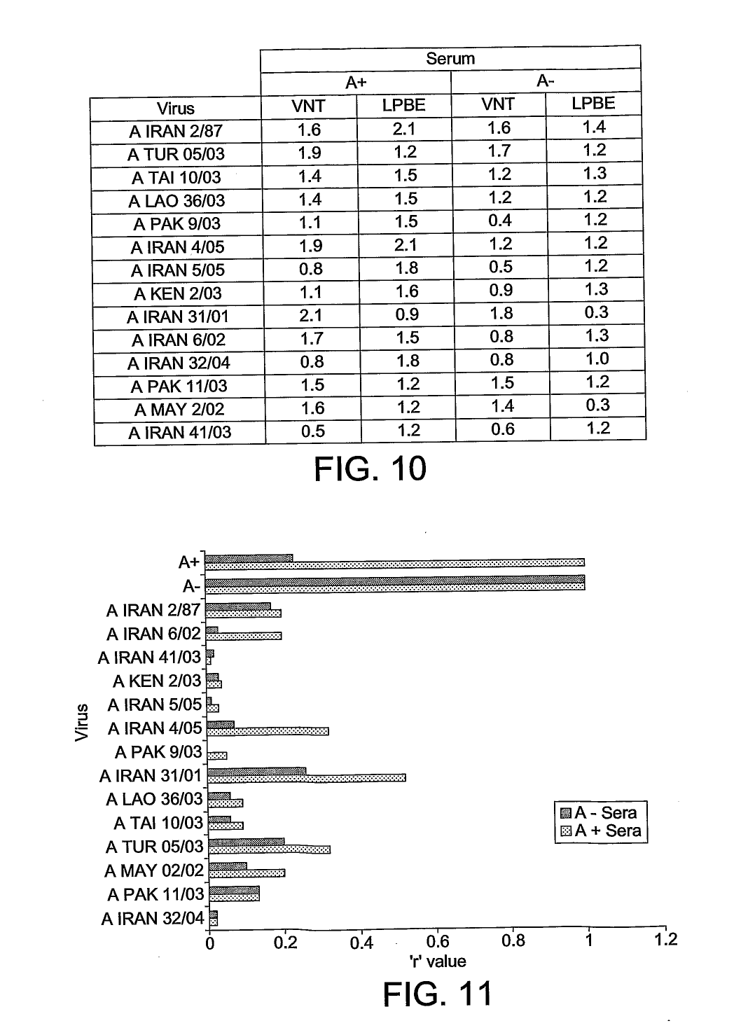

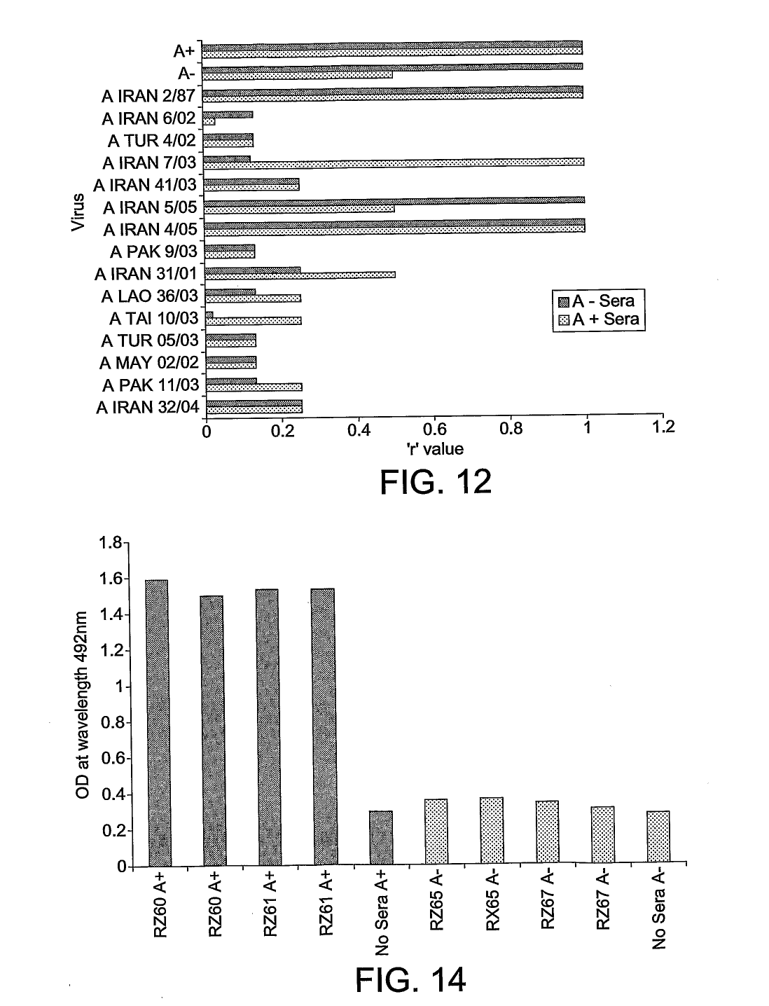

The usefulness of A− as a vaccine virus compared to A+ was investigated against a panel of field isolates. VNT and LPBE titres presented in FIG. 10 were used to calculate relationship values which are presented in FIGS. 11 and 12.

As shown in FIG. 10, the A+ serum, consistent with other sections in this thesis, is generally better at neutralising field isolates than A− serum. This is mirrored by r values presented in FIG. 11, which show that A+ has an overall greater relationship with field isolates than A− serum. This same phenomenon can be seen when the LPBE sera titres are considered (FIG. 10). Although the LPBE serum titres are relatively low for both A+ and A− against the field isolates, the A+ serum is in the main, more efficient at blocking the binding of the guinea pig polyclonal detection antibody than the A− sera.

With reference to FIGS. 11 and 12, and interpretation of the r values, A+serum generally has a greater relationship with field isolates than A− serum by virus neutralisation test, however, r values calculated from the LPBE titres suggest otherwise. In twelve out of seventeen cases, the r values are either, identical between A+ and A− serum (A+, AIRAN 2/87, A TUR 4/02, A IRAN 41/03, A IRAN 4/05, A PAK 9/03, A TUR 05/03, A MAY 02/02 and A IRAN 32/04) when compared against a range of viruses, or improved above those calculated for A+(A−, A IRAN 6/02, and A IRAN 5/05).

### Example 7

### Investigation as to Whether A− Vaccine can be Used as a Marker Vaccine

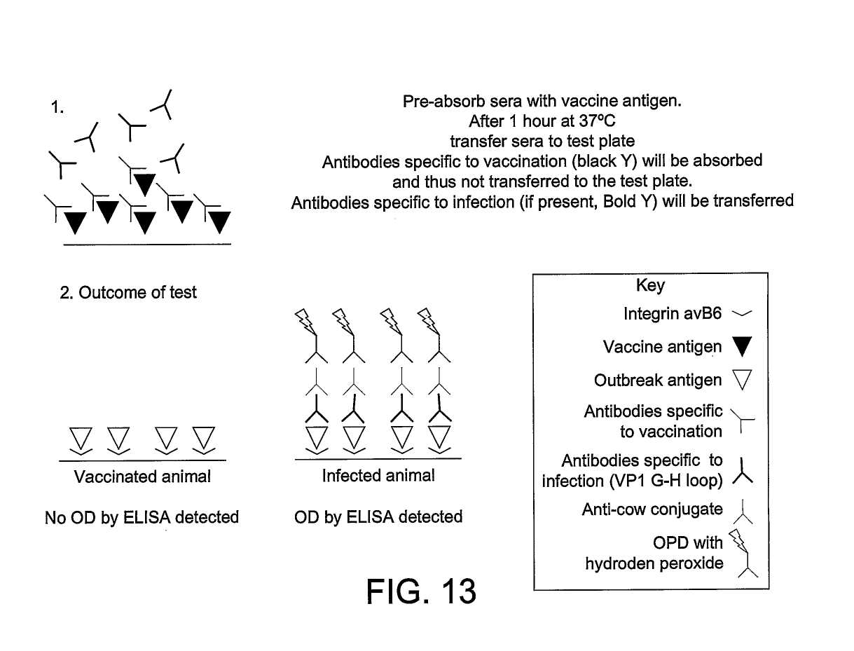

In order to determine whether a vaccine derived from A− virus could be used as a negative marker vaccine, pooled serum was first screened in order to determine the optimum serum dilution at which is was possible to discriminate effectively between vaccinated (A− serum) animals and ‘theoretically infected’ (A+ serum) animals. A serum dilution of 1 in 80 was chosen to repeat the test using individual serum samples from A+ and A− vaccinated cattle, as this was the serum titre which gave the largest difference between A+ and A−. For differentiation analysis two animals were randomly selected from either the A+ vaccinated group (RZ60 and 61) and the A− vaccinated group (RZ65 and 67). Pre-absorbing A− serum at 1 in 80 with FMDV A− antigen (1 μg/ml) efficiently removed the vaccination related antibody response demonstrated by the A− serum values being identical to the no serum negative control (FIG. 13). Absorbing A+ serum with FMDV A− antigen was not able to remove all the response due to the A+ serum containing antibodies to antigenic sites not found on A− virus, therefore making it easy to discriminate between vaccinated (A− serum) and theoretically infected (A+ serum) animals (FIG. 14).

### Example 8

### Cattle Challenge Experiment

Twelve cattle, 6-9 months of age are divided into three groups: two groups of 5 vaccinated subjects and one group of two unvaccinated subjects.

Cattle are vaccinated with 1 bovine dose of 15 ug of A+(first group) and 1 bovine dose of 15 ug of A− (second group). The third group of two steers are not vaccinated.

All cattle are challenged on day 21 post challenge with 10,000 ID₅₀/10⁵ TCID₅₀ cattle adapted A+.

### Discussion

Collectively the VNT and IgG titre analyses suggest the following:

(i) both A+ and A− sera can bind to and neutralise their homologous antigens to comparable titres;
(ii) A+ serum binds equally well to both A+ and A− virus but neutralises A− virus at higher serum dilutions; and
(iii) in contrast, A− serum preferentially binds and neutralises its homologous virus and can only bind and neutralise A+ virus at lower serum dilutions.

Based on the above observations, one might predict that the A− vaccine would not be as effective at protecting cattle from challenge as a wild type vaccine virus. However, r values calculated from serum titres did not support this. The mean serum titre from this group of cattle was 1.8 log 10 which with reference to Barnett et al., ((2003) Vaccine. 21. 3240-3248) and serotype A, indicate a probability of protection approximating 0.8. In addition, r values calculated from virus neutralising antibody titres, indicate that, three out of the five animals have antibody titres which would be considered likely to achieve protection (Paton et al., (2005) Revue scientifique et technique (International Office Epizootics). 24. 981-993) (table 6.5), whereas, r values calculated from IgG antibody titres, indicate that, four out of the five animals have antibody titres which would be considered likely to achieve protection (table 6.7). The lower amount of animals predicted to be protected by use of virus neutralising antibody titres can be explained by the VNT test itself. The VNT test by its nature, is preferentially selecting a certain class of antibody, namely those that block the virus from attaching to its cellular receptor. It is known that VP1 G-H loop peptides and VP1 G-H loop specific MAbs can in the absence of any other antibody block virus attachment to cellular receptors and neutralise the virus. Therefore a serum which contains antibodies which are cross-reactive to the VP1 G-H loop and can prevent viral attachment, would offer greater potential for in vitro neutralisation than a serum which does not contain these antibodies. In this case, A− serum does not contain antibodies to the VP1 G-H loop, therefore compromising its ability to neutralise wild type viruses such as A+ in vitro.

In any case, the fact that A− can measurably neutralise and bind A+ virus is highly significant, as this demonstrates (i) other mechanisms of neutralisation such as viral aggregation and/or conformational alteration of the capsid structure leading to the release of viral RNA (McCullough et al., (1987) Immunology. 60. 75-8) are working effectively; and (ii) the A− antibodies can still bind to other antigenic sites on A+. This is particularly true in view of the knowledge that IgG antibody-enhanced opsonisation and subsequent phagocytosis is thought to be more important for in vivo protection (McCullough et al., (1988) Immunology. 65. 187-191 and reviewed in McCullough et al., (1992) Journal of Virology. 66. 1835-1840).

Finally, from the r values calculated from the LPBE serum titres (FIG. 9) it is predicted that all cattle vaccinated with A− vaccine virus would be protected against challenge with wild type challenge. This provides strong evidence that a vaccine lacking the VP1 G-H loop would be capable of protecting cattle from wild type challenge.

The serum from A+ and A− vaccinated cattle was compared to a random selection of A serotype field isolates to determine whether the loss of the VP1 G-H loop had compromised the serums' relationship. In fact, it was found that in many cases A− serum r values were virtually identical to those calculated for A+ serum, showing that no compromise had occurred (FIG. 11).

Interestingly and in support of Frimann's published work (Frimann et al., (2007) Vaccine. 25. 6191-6200), it was found that the binding relationship had been improved above that of A+ serum in three cases, A−, A IRAN 6/02 and A IRAN 5/05. As explained in the Background section, the G-H loop of VP1 is both a dominant and a variable site. This variability is potentially a problem in vaccine design because it necessitates a close match between vaccine strain and virus. It may be that removal of the G-H loop in the A− strain effectively “silences” this immunodominant epitope, causing the immune response to be diverted towards less dominant but more conserved, protective epitopes. Hence, it is possible that this ‘improvement’ in reactivity observed with some field isolates occurred due to sequence homology in the backbone of the virus which was shared between A− and the viruses. Since the A− serum is not ‘contaminated’ with less well matched VP1 G-H loop antibodies, it may be that the more specific backbone antibodies are able to bind more efficiently.

As backbone identities between the vaccine virus and field isolate are greater than VP1 G-H loop similarities, the loop-negative vaccine of the present invention shows an improvement over traditional loop-positive vaccines. In this respect, the vaccine of the invention stimulates an enhanced non-VP1 G-H loop antibody response giving improved vaccine efficacy to heterologous virus which more than compensated for the decrease in anti-VP1 G-H loop response.

### Materials and Methods

### Viruses and Antigen Production

Two viruses, A− with a 13 amino acid deletion within the VP1 G-H loop and A+ with the native VP1 G-H loop were used in this study. Viruses A− and A+ were obtained from Nick Knowles, Institute for Animal Health, Pirbright Laboratory. The viruses were supplied as tissue culture supernatants which had been harvested following at least three plaque purifications. On receipt, both viruses were passaged once in BHK cells (175 cm² flasks) as described in section 2.4.3 in order to increase the virus titre and volume. Clarified virus supernatant from the BHK passage was used to inoculate 20 rollers (1700 cm²), 10 per virus type. On appearance of 100% CPE, both viruses were harvested, BEI inactivated and sucrose density gradient purified as described in section 2.7.3.10% of the clarified supernatant was kept as live virus and was stored at −70° C. for in vitro assays.

### Vaccine Formulation

Vaccine antigen was concentrated and purified from viruses A− and A+. Fractions collected following sucrose density gradient purification were analysed by UV light absorbance to determine the most suitable for vaccine production. The quantity of antigen was calculated and 10 bovine doses of vaccine were formulated at 15 μg per 2 ml dose.

### Animals

Ten Holstein Friesian cross-bred steers were ear tagged and housed in two groups of five within ISO 9 (‘clean’ isolation unit used for non-infectious purposes only).

### Vaccine and Vaccine Trial

Two water-in-oil-in-water vaccines were prepared as described in section 2.6.5. Cattle RZ59-63 received the A+ derived vaccine while cattle RZ 64, 65, 67, 68 and 81 received the A− derived vaccine. Cattle were intramuscularly inoculated with 2 ml of the appropriate vaccine into their neck as described in section 2.7.8. Blood samples were taken on the days detailed in section 2.7.8. 10 ml of clotted and heparinised blood were collected via the appropriate vacutainer on each occasion, with the exception of day 21, where 120 ml of clotted blood was collected using a 60 ml syringe and 500 ml glass bottles.

### Serology

VNT's and VT's were carried out using standard procedures. MAb's used in this study have been previously characterised and their footprints have been mapped to include residues 138-154 of VP1 (Bolwell et al., (1989) as above). Individual sera were used to screen against A+ and A− viruses, pooled sera were used against field isolates. All antibody titres were converted to log₁₀.

In order to determine whether it was possible to use A− virus as a marker vaccine, an indirect integrin ELISA was performed and carried out as follows. In the absence of being able to experimentally challenge the A− vaccinated cattle, A+ serum was considered as an ‘infected’ animal serum since it contains antibodies to antigenic sites not found in A− virus, whereas A− serum was considered as the ‘vaccinated’ animal serum. Animals which were theoretically infected (A+ serum) should give a strong OD whereas animals that had just been vaccinated (A− serum) should have OD values comparable to no sera control wells. Integrin αvβ6 was diluted to 0.2 μg/ml in integrin coating buffer and added to 96 well micro titre plates (maxisorb immunoplates, Nunc) (50 μl/well). Following overnight incubation at 4° C., integrin blocking buffer was added at 50 μl/well. After 1 hour, FMDV antigen (A+) was added at 1 μg/ml (diluted in blocking buffer, 50 μl/well) and the plate incubated for 1 hour. At the same time, day 21 pooled sera from A+ and A− vaccinated cattle were diluted to 1 in 20 in blocking buffer (50 μl/well) and titrated 2 fold down a cell culture plate. FMDV antigen A− was then added to the serum at 1 μg/ml (diluted in blocking buffer, 50 μl/well) and incubated for 1 hour. Following incubation, 50 μl of the serum/A− antigen mix was added to the A+ antigen coated plate which had been washed three times with PBS and incubated for 1 hour. One row was left as a no serum control whereby only integrin blocking buffer was added. After a further hour, peroxidase conjugated anti-bovine antibody (SIGMA), diluted 1/5000 in integrin blocking buffer, was added to the plates. The plates were again incubated for 1 hour before being developed with OPD substrate. After 10 minutes plates were stopped with 1.25M sulphuric acid and absorbances read at 492 nm on a spectrophotometer (Dynex Technologies).

### Sequencing of Viruses

A+ and A− were sequenced through the VP1 G-H loop to confirm that the VP1 G-H loop was retained in A+ and that the spontaneous deletion remained in A− following serial passage on BHK cells. RNA was extracted from BHK culture supernatant and the RT performed. The VP1 coding region was obtained by PCR utilising primers pA-1C621F and pNK72 which have the following sequences:

pA-1C612F TAGCGCCGGCAAAGACTTTGA
pA-1C562F TACCAAATTACACACGGGAA
pNK72 GAGTCCAACCCTGGGCCCTTC
pNK61 GACATGTCCTCCTGCATCT.

The VP1 coding region from A+ and A− virus were then sequenced using primers pA-1C562F and NK61. Sequence data for the rest of the capsid of A+ and A− were obtained from Nick Knowles, Institute for Animal Health, Pirbright Laboratory.

### Statistical Analysis

A two sample t-test was used to determine the statistical significance between the homolgous IgG titres (FIG. 6), VNT titres (FIG. 4) and the LPBE titres (FIG. 8). A paired t-test was used to calculate the significance of across column analysis, whilst a two-sample t-test was used to calculate the significance of down column analysis. Statistical analysis was performed using Minitab version 14.15.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in virology, or related fields are intended to be within the scope of the following claims.

1. A foot and mouth disease (FMD) vaccine comprising a vaccinating entity comprising or capable of expressing a foot and mouth disease virus (FMDV) VP1 polypeptide having a deletion of at least seven amino acids in the G-H loop such that the VP1 polypeptide lacks an RGD motif.
wherein the vaccine is substantially free from a vaccinating entity comprising or capable of expressing an FMDV VP1 polypeptide having a complete G-H loop.

2. A vaccine according to claim 1, which consists essentially of the vaccinating entity comprising or capable of expressing a foot and mouth disease virus (FMDV) VP1 polypeptide having a deletion in the G-H loop.

3. A vaccine according to claim 1, wherein the vaccinating entity is an activated FMD virus.

4. A vaccine according to claim 1, wherein the vaccinating entity is an attenuated form of FMDV.

5. A vaccine according to claim 1, wherein the vaccinating entity is a nucleotide sequence encoding the FMD proteins needed to produce an FMDV empty capsid particle.

6. A vaccine according to claim 1, wherein the vaccinating entity comprises or is capable of expressing a serotype A VP1 polypeptide having a deletion of residues 142-154 in the G-H loop.

7. (canceled)

8. A method for preventing FMD in a subject which comprises the step of administering an effective amount of a vaccine according to claim 1 to the subject.

9-10. (canceled)

11. A method for distinguishing between
a) FMD infected, sub-clinically infected or previously infected subjects; and
b) subjects vaccinated with a vaccine according to claim 1, which comprises the step of investigating the presence of a VP-1 polypeptide comprising a G-H loop, or evidence of an immune response against the VP-1 G-H loop in the subject;
the presence of a VP-1 polypeptide comprising a G-H loop, or evidence of an immune response against the VP-1 G-H loop in the subject, indicating that the subject is or has been infected with FMDV.

12. A method according to claim 11, which comprises the step of investigating the presence of antibodies to the G-H loop in the subject, which step involves:
i) removal of non-G-H loop antibodies in a sample from the subject;
ii) detecting the presence of antibodies after the removal step
the presence of antibodies after the removal step indicating the presence of G-H loop antibodies in the subject.

13. A method according to claim 11, which comprises the step of investigating the presence of a VP-1 polypeptide comprising a G-H loop using an ανβ6 integrin.

14. A differentiation assay to distinguish between
a) FMD infected, sub-clinically infected or previously infected subjects; and
b) subjects vaccinated with a vaccine according to claim 1, which comprises a detection system for VP-1 GH loops or for anti-G-H loop antibodies.

15. A differentiation assay according to claim 14, wherein the detection system detects anti-G-H loop antibodies, which includes a vaccinating entity for pre-absorption of non-G-H loop antibodies, said vaccinating entity comprising a foot and mouth disease virus (FMDV) VP1 polypeptide having a deletion of at least seven amino acids in the G-H loop such that the VP1 polypeptide lacks an RGD motif,
wherein the vaccine is substantially free from a vaccinating entity comprising or capable of expressing an FMDV VP1 polypeptide having a complete G-H loops.

16. A differentiation assay according to claim 14, which comprises an ανβ6 integrin for detection of VP-1 GH loops.

17. A kit which comprises a vaccine comprising a vaccinating entity comprising or capable of expressing a foot and mouth disease virus (FMDV) VP1 polypeptide having a deletion of at least seven amino acids in the G-H loop such that the VP1 polypeptide lacks an RGD motif,
wherein the vaccine is substantially free from a vaccinating entity comprising or capable of expressing an FMDV VP1 polypeptide having a complete G-H loop, together with a differentiation assay according to claim 14.

18. An immunogenic preparation comprising a polypeptide and one or more adjuvants, excipients, carriers, or diluents, wherein the polypeptide is a foot and mouth disease virus (FMDV) VP1 polypeptide lacking from 7 to 21 amino acids in the VP1 G-H loop, such that the polypeptide lacks an RGD motif.

19. An immunogenic preparation according to claim 18, wherein the preparation contains an inactivated, dead, or attenuated FMDV, and wherein the FMDV contains the VP1 polypeptide.

20. An immunogenic preparation comprising a polynucleotide that encodes a foot and mouth disease virus (FMDV) VP1 polypeptide lacking from 7 to 21 amino acids in the VP1 G-H loop, such that the polypeptide lacks an RGD motif.

21. A method of impeding contraction of foot and mouth disease (FMD) in a mammal susceptible to FMD, the method comprising administering a composition comprising a foot and mouth disease virus (FMDV) VP1 polypeptide lacking from 7 to 21 amino acids in the VP1 G-H loop, such that the polypeptide lacks an RGD motif.
